(19) United States
(12) Patent Application Publication
(10) Pub. No.: US 2026/0185158 A1
(43) Pub. Date: Jul. 2, 2026

(54) METHODS FOR GENERATING UNIQUE MOLECULAR IDENTIFIERS AND USES THEREOF

(71) Applicant: Illumina, Inc., San Diego, CA (US)

(72) Inventors: Michael Shane Smith, San Diego, CA (US); Eric Hans Vermaas, San Diego, CA (US)

(21) Appl. No.: 19/432,616

(22) Filed: Dec. 24, 2025

Related U.S. Application Data
(60) Provisional application No. 63/738,995, filed on Dec. 26, 2024.

(51) Int. Cl. C12Q 1/6874; C12Q 1/34; C12Q 1/6806; C12Q 1/6855; C12Q 1/686

(52) U.S. Cl. CPC C12Q 1/6874; C12Q 1/34; C12Q 1/6806; C12Q 1/6855; C12Q 1/686; G01N 2333/922

(57) ABSTRACT

The disclosure provides methods, compositions, and kits for amplifying and sequencing double-stranded nucleic acids using adapters including landmark sequences that are uniquely associated with individual polynucleotide fragments. In some aspects, the landmark sequences include a plurality of nucleotide analogs.

### CROSS-REFERENCE TO RELATED APPLICATIONS

This application claims priority benefit of U.S. Provisional Application No. 63/738,995, filed Dec. 26, 2024, which is hereby incorporated by reference in its entirety.

### BACKGROUND

Next generation sequencing technology is providing increasingly high speed of sequencing, allowing larger sequencing depth. However, because sequencing accuracy and sensitivity are affected by errors and noise from various sources, e.g., sample defects, PCR during library preparation, enrichment, clustering, and sequencing, increasing depth of sequencing alone cannot ensure detection of sequences of very low allele frequency, such as in fetal cell-free DNA (cfDNA) in maternal plasma, circulating tumor DNA (ctDNA), and sub-clonal mutations in pathogens. Therefore, it is desirable to develop methods for determining sequences of DNA molecules in small quantity and/or low allele frequency while suppressing sequencing inaccuracy due to various sources of errors.

### SUMMARY

The present disclosure is directed to methods, compositions, and kits for amplifying and sequencing double-stranded nucleic acids using adapters comprising landmark sequences. In some aspects, the landmark sequences comprise a plurality of nucleotide analogs.

Some aspects of the present disclosure are directed to a method for amplifying a double-stranded nucleic acid molecule, the method comprising: a. attaching adapters to both ends of a target double-stranded nucleic acid molecule, thereby generating a double-stranded nucleic acid molecule comprising a first strand adapter-target nucleic acid sequence and a second strand adapter-target nucleic acid sequence, wherein each adapter comprises a double-stranded region, and wherein each strand of the double-stranded region comprises a landmark sequence comprising a plurality of nucleotide analogs; b. annealing a first primer to each of the first strand adapter-target nucleic acid sequence and the second strand adapter-target nucleic acid sequence; c. extending the annealed first primers with a polymerase, thereby generating a first double-stranded extension product comprising a complement of the first strand adapter-target nucleic acid, and a second double-stranded extension product comprising a complement of the second strand adapter-target nucleic acid sequence, wherein each of the complement of the first strand adapter-target nucleic acid sequence and complement of the second strand adapter-target nucleic acid sequence comprises a landmark sequence complement; d. annealing a second primer to each of the complement of the first strand adapter-target nucleic acid sequence and the complement of the second strand adapter-target nucleic acid sequence; and e. amplifying the complement of the first strand adapter-target nucleic acid sequence and the complement of the second strand adapter-target nucleic acid sequence with a polymerase, thereby generating a plurality of first amplification products and a plurality of second amplification products.

In some aspects, unincorporated first primers (i.e., first primers which were not extended by the polymerase in step c.) are removed after step c. In some aspects, the unincorporated first primers of step c are removed before step d. In some aspects, the unincorporated first primers are removed by single-stranded nuclease treatment, chromatography, or ultrafiltration. In some aspects, the unincorporated first primers are removed by single-stranded nuclease treatment. In some aspects, following single-stranded nuclease treatment, the first double-stranded extension product and the second double-stranded extension product of step c are purified. In some aspects, the single-stranded nuclease is S1 nuclease, P1 nuclease, or exonuclease VII.

In some aspects, the unincorporated first primers are removed by chromatography. In some aspects, the chromatography comprises size exclusion chromatography or immobilized metal affinity chromatography (IMAC). Examples of IMAC removal of unincorporated primers using, e.g., Cu²⁺ iminodiacetic acid agarose are described in Kanakaraj I et al. PLOS One. 2011; 6(1): e14512. In some aspects, the unincorpoated primers are removed by ultrafiltration. In some aspects, the ultrafiltration comprises membrane filtration.

In some aspects, the first primer comprises an affinity tag at the 5′ end. In some aspects, the affinity tag is a biotin moiety. In some aspects, the first and second double-stranded extension products of step c each comprise the affinity tag (e.g., each comprise a biotin moiety at a 5′ end). In some aspects, after step c, the method firther comprises contacting the first double-stranded extension product comprising the affinity tag and the second double-stranded extension product comprising the affinity tag with a solid support comprising an immobilized capture agent (e.g., an avidin or strepavidin moiety), wherein the immobilized capture agent binds to the affinity tag of each double-stranded extension product. In some aspects, the immobilized capture agent comprises an avidin moiety. In some aspects, the immobilized capture agent comprises a strepavidin moiety. In some aspects, after binding of each double-stranded extension product to the solid support, the double-stranded nucleic acids are denatured, and the first strand adapter-target nucleic acid sequence and second strand nucleic acid sequence (i.e., the strands that are not bound to the solid support) are removed.

In some aspects, the adapters comprise an affinity tag (e.g., a biotin moiety) at a 5′ end (i.e., at a 5′ end of one of the polynucleotide strands of the adapter). In some aspects, the method further comprises, after step a, contacting the double-stranded nucleic acid molecule of step a to a solid support comprising an immobilized capture agent (e.g., an avidin or strepavidin moiety), wherein the immobilized capture agent binds to the affinity tag of the adapters. In some aspects, step b comprises annealing a first primer to each of the immobilized first strand adapter-target nucleic acid sequence and the immobilized second strand adapter-target nucleic acid sequence. In some aspects, after step c, the double-stranded nucleic acids are denatured, and the complement of the first strand adapter-target nucleic acid sequence and the complement of the second strand nucleic acid sequence (i.e., the strands of the double-stranded nucleic acids that are not bound to the solid support) are eluted and subjected to steps d and e.

In some aspects, the adapters are Y-shaped adapters comprising a single-stranded 5′ arm, a single-stranded 3′ arm, and a double-stranded region comprising the landmark sequence. In some aspects, the single-stranded 3′ arm comprises a capture sequence (see, e.g., FIG. 3A). In some aspects, the 3′ end of the single-stranded region is blocked (e.g., the 3′ end does not comprise a 3′-OH) to prevent extension. In some aspects, the single-stranded 5′ arm comprises a primer binding sequence (e. g, a first primer binding sequence). In some aspects, the double-stranded region of the Y-shaped adapter further comprises an index sequence. In some aspects, the adapters comprise a first plurality of Y-shaped adapters and a second plurality of Y-shaped adapters. In some aspects, the double-stranded region of the first plurality of Y-shaped adapters comprise a first index sequence and the second plurality of Y-shaped adapters comprise a second index sequence. In some aspects, the indeces are used for sample identification (i.e., identification of individual samples from a plurality of samples). In some aspects, the indeces are used for strand identification (i.e., identification and/or tracking of each strand of the double-stranded nucleic acid). In some aspects, the indeces are used for error correction. Exemplary methods for using indeces for error correction are described in, e.g., U.S. Pat. Nos. 10,844,428 and 10,844,429, each of which is incorporated herein by reference.

In some aspects, the double-stranded region of the first plurality of Y-shaped adapters comprise a first landmark sequence and the second plurality of Y-shaped adapters comprise a second landmark sequence. In some aspects, step a comprises attaching the Y-shaped adapters to both ends of the target double-stranded nucleic acid molecule. In some aspects, step a comprises attaching the first plurality of Y-shaped adapters and the second plurality of Y-shaped adapters to each end of the target double-stranded nucleic acid molecule, respectively, thereby generating a double-stranded nucleic acid molecule comprising a Y-shaped adapter of the first plurality at one end and a Y-shaped adapter of the second plurality at the second end (see, e.g., FIG. 3B). In some aspects, the first strand of the double-stranded nucleic acid molecule (i.e., the first strand adapter-target nucleic acid sequence) comprises, from 5′ to 3′, a first primer binding sequence, a first index sequence, a first landmark sequence complement, a target nucleic acid sequence, a second landmark sequence, a second index sequence complement, and a capture sequence. In some aspects, the second strand of the double-stranded nucleic acid molecule (i.e., the second strand adapter-target nucleic acid sequence) comprises, from 5′ to 3′, a first primer binding sequence, a second index sequence, a second landmark sequence complement, a target nucleic acid sequence complement, a first landmark sequence, a first index sequence complement, and the capture sequence.

In some aspects, the method further comprises hybridizing the double-stranded nucleic acid molecule comprising the Y-shaped adapters to a solid support comprising immobilized capture oligonucleotides, wherein the immobilized capture oligonucleotides are complementary to the capture sequence of the Y-shaped adapter, thereby forming double-stranded nucleic acid molecules complexes. In some aspects, the immobilized capture oligonucleotides comprise, from 5′ to 3′, a cleavage site, a primer binding sequence (e.g., a second primer binding sequence) and a capture sequence complement (i.e., a sequence complementary to the capture sequence of the Y-shape adapters). In some aspects, the immobilized capture oligonucleotides further comprises a barcode sequence (e.g., a bead index sequence or a cellular barcode sequence). In some aspects, the immobilized capture oligonucleotides comprise, from 5′ to 3′, the cleavage site, a second primer binding sequence, the barcode sequence, and the capture sequence complement (see, e.g., FIG. 3C).

In some aspects, the method further comprises extending the immobilized capture oligonucleotides of the double-stranded nucleic acid molecule complexes, thereby generating a first immobilized extended capture oligonucleotide and a second immobilized extended capture oligonucleotide (see, e.g., FIG. 3D). In some aspects, the first immobilized extended capture oligonucleotide comprises, from 5′ to 3′, the cleavage site, the second primer binding sequence, the capture sequence complement, the first index sequence, the first landmark sequence complement, the second landmark sequence, the second index sequence complement, and a first primer binding sequence complement. In some aspects, the second immobilized extended capture oligonucleotide comprises, from 5′ to 3′, the cleavage site, the second primer binding sequence, the capture sequence complement, the second index sequence, the second landmark sequence complement, the first landmark sequence, the first index sequence complement, and a first primer binding sequence complement. In some aspects, the first immobilized extended capture oligonucleotide and the second immobilized extended capture oligonucleotide each comprise a barcode sequence.

In some aspects, following the extending, unbound nucleic acids (e.g., unbound double-stranded nucleic acid molecules) are removed from the solid support (e.g., removed by denaturation and/or washing). In some aspects, the method further comprises contacting the cleavage site of the first and second immobilized extended capture oligonucleotides with a cleaving agent, thereby releasing the first and second immobilized extended capture oligonucleotides from the solid support. In some aspects, the cleaving agent is an enzymatic cleaving agent or a chemical cleaving agent. In some aspects, the enzymatic cleaving agent is a restriction endonuclease. In some aspects, the method further comprises amplifying the released first and second immobilized extended capture oligonucleotides. In some aspects, the amplification comprises hybridizing a first primer to the first primer binding sequence complement and a second primer to the second primer binding sequence, and extending the first and second primers with a polymerase. In some aspects, the method further comprises sequencing the amplified first and second immobilized extended captures oligonucleotides.

In some aspects, the plurality of first amplifications products is distinguishable from the plurality of second amplification products.

In some aspects, the plurality of first amplification products comprises a sequence of the first strand of the target double-stranded nucleic acid molecule, and complement thereof, and wherein the plurality of second amplification products comprises the second strand of the target double-stranded nucleic acid molecule, and complement thereof.

In some aspects, the landmark sequence complement does not comprise nucleotide analogs.

In some aspects, each landmark sequence comprises 3 or more nucleotide analogs. In some aspects, each landmark sequence comprises 6 or more nucleotide analogs.

In some aspects, the nucleotide analogs are not adjacent to each other.

In some aspects, each of the nucleotide analogs is separated by at least 3 nucleotides. In some aspects, each of the nucleotide analogs is separated by at least 5 nucleotides.

In some aspects, the nucleotides analogs comprise one or more degenerate bases. In some aspects, the one or more degenerate bases comprise inosine, the pyrimidine base 6H,8H-3,4-dihydropyrimido[4,5-c][1,2]oxazin-7-one (P), the purine base N⁶-methoxy-2,6-diaminopurine (K), 5-nitroindole, or any combination thereof. In some aspects, when the one or more degenerate bases comprise inosine, step c of the method is performed in the presence of a nucleotide mixture comprising uracil, adenine, thymine, cytosine, and guanine. In some aspects, the molar concentration of cytosine is reduced in comparison to the molar concentration of the other nucleotides of the nucleotide mixture. In some aspects, the molar concentration of each nucleotide in the nucleotide mixture is adjusted to promote alternate base pairing with the one or more degenerate bases. In some aspects, the concentration of each nucleotide in the nucleotide mixture is adjusted to promote alternate base pairing with inosine (see, e.g., Licht K et al. Nucleic Acids Res. 2019; 47(1):3-14). Additional disclosure and methods of using degenerate bases is described in, e.g., Loakes D and Brown D M. Nucleic Acids Res. 1994; 22(2):4039-43; Lin P K and Brown D M. Nucleic Acids Res. 1989; 17(24): 10373-83; Lin P K and Brown D M. Nucleic Acids Res. 1992; 20:5149-52; and Liu H and Nichols R. Biotechniques. 1994; 16:24-26, each of which is incorporated herein by reference in its entirety.

In some aspects, the first strand of the adapter comprises a first landmark sequence and wherein the second strand of the adapter comprises a second landmark sequence. In some aspects, the first landmark sequence and the second landmark sequence are substantially complementary. In some aspects, the first landmark sequence consists of a first exogenous landmark sequence and the second landmark sequence consists of a second exogenous landmark sequence, wherein each of the exogenous landmark sequences comprises one or more nucleotide analogs. In some aspects, the first landmark sequence is adjacent to a first endogenous landmark sequence and the second landmark sequence is adjacent to a second endogenous landmark sequence, wherein each endogenous landmark sequence corresponds to a breakpoint in the target double-stranded nucleic acid molecule. In some aspects, the plurality of first amplification products is related to the plurality of second amplification products by the first exogenous landmark sequence, the first endogenous landmark sequence, or a combination thereof.

In some aspects, the first primer comprises a sequence complementary to a region of each of the first strand adapter-target nucleic acid sequence and the second strand adapter-target nucleic acid sequence 3′ of the landmark sequence.

In some aspects, the first primer comprises a first index sequence and one or more primer binding sequences.

In some aspects, the second primer comprises a second index sequence and one or more primer binding sequences.

In some aspects, each adapter comprises a single-stranded 5′ arm and a single-stranded 3′ arm. In some aspects, the first primer is complementary to a portion of the single-stranded 3′ arm. In some aspects, the second primer is complementary to a portion of the complement of the single-stranded 5′ arm.

In some aspects, each adapter further comprises a random sequence. In some aspects, the random sequence is between 2 to 8 nucleotides in length.

In some aspects, the adapter attached to the first strand adapter-target nucleic acid sequence and the adapter attached to the second strand adapter-target nucleic acid sequence comprise different landmark sequences.

In some aspects, the adapter is between 25 and 80 nucleotides in length. In some aspects, the adapter is greater than 80 nucleotides in length. In some aspects, the adapter is about 25, about 40, about 50, about 60, about 70, or about 80 nucleotides in length.

In some aspects, the amplifying of step (e) comprises PCR amplification.

In some aspects, prior to step (b), the method comprises denaturing the double-stranded nucleic acid molecule. In some aspects, the denaturing comprises chemical denaturation, thermal denaturation, or both chemical and thermal denaturation.

In some aspects, prior to step (d), the method comprises denaturing the first and second double-stranded extension products.

In some aspects, the method further comprises step (f) sequencing the plurality of first amplification products, thereby generating a plurality of first sequence reads, and sequencing the plurality of second amplification products, thereby generating a plurality of second sequence reads. In some aspects, the method further comprises step (g) comparing at least one sequence of the plurality of first sequence reads with at least one sequence obtained from the plurality of second sequence reads, thereby generating a consensus sequence of the double-stranded target nucleic acid molecule. In some aspects, generating the consensus sequence comprises grouping the at least one sequence of the plurality of first sequence reads with at least one sequence obtained from the plurality of second sequence reads is based at least on the landmark sequence, or complement thereof.

In some aspects, prior to step (a), the adapter comprises a 3′ overhang.

In some aspects, prior to step (a), the adapter is blunt-ended.

In some aspects, the double-stranded nucleic acid molecule comprises DNA, RNA, or both DNA and RNA. In some aspects, the double-stranded nucleic acid molecule consists of DNA. In some aspects, the DNA is genomic DNA or cDNA derived from RNA.

In some aspects, the double-stranded nucleic acid molecule is isolated from a biological sample. In some aspects, the biological sample is a single cell or a tissue sample.

In another aspect, the present disclosure provides a kit comprising a plurality of the adapters, a plurality of the first primers, and a plurality of the second primers as described herein. In some aspects, the kit further comprises one or more polymerases. In some aspects, the kit further comprises a solid support comprising an immobilized capture agent. In some aspects, the kit further comprises a solid support comprising an immobilized capture oligonucleotide. In some aspects, the immobilized capture oligonucleotide comprises, from 5′ to 3′, a cleavage site, a primer binding sequence, and a capture sequence complement. In some aspects, the immobilized capture oligonucleotide futher comprises a barcode sequence.

In another aspect, the present disclosure provides a solid support comprising a first immobilized extended capture oligonucleotide and a second immobilized extended capture oligonucleotide, wherein the first immobilized extended capture oligonucleotide comprises a cleavage site, a first primer binding sequence, a capture sequence complement, a first index sequence, a first landmark sequence complement, a second landmark sequence, a second index sequence complement, and a second primer binding sequence complement. In some aspects, the second immobilized extended capture oligonucleotide comprises the cleavage site, the first primer binding sequence, the capture sequence complement, a second index sequence, a second landmark sequence complement, a first landmark sequence, a first index sequence complement, and a second primer binding sequence. In some aspects, the cleavage site is a chemical cleavage site or an enzymatic cleavage site.

### BRIEF DESCRIPTION OF THE DRAWINGS

FIG. 1 illustrates a flow chart of an exemplary workflow using landmark sequences to sequence nucleic acid fragments.

FIGS. 2A-2D show the steps of an exemplary workflow using landmark sequences to uniquely identify individual nucleic acid strands from a double-stranded nucleic acid fragment. FIG. 2A shows the step of ligating landmark sequence (LIS)-containing adapters to an end-repaired and A-tailed double-stranded nucleic acid fragment. The top strand of the double-stranded nucleic acid fragment is labeled with as ‘α’ and the bottom strand as ‘β’. ME represents a mosaic end transposase recognition sequence. A14 and B15′ are primer binding sites. FIG. 2B shows the steps of denaturing the adapter-ligated double-stranded nucleic acid, annealing first indexing primers, and performing a linear amplification. FIG. 2C shows the product of the linear amplification, wherein the complement of the landmark sequence (LIS) is shown as LLS′, and the complement of LIS′ is shown as LLS. A linear or exponential amplification reaction is then performed using second indexing primers. FIG. 2D shows the product of the amplification reaction, which may subsequently be used in a sequencing workflow.

FIGS. 3A-3D show various oligonucleotide construcs for use in the methods disclosed herein. FIG. 3A shows Y-shaped adapters comprising a primer binding sequence (PBS 1), an index sequence (index 1 or index 2), a landmark sequence (LIS 1 or LIS 2), and a capture sequence. FIG. 3B shows a double-stranded nucleic acid with the Y-adapters of FIG. 3A attached to each end. FIG. 3C shows immobilized capture oligonucleotides comprising a cleavage site, a primer binding sequence (PBS 2), a barcode sequence, and a capture sequence complment. FIG. 3D shows immobilized extended capture oligonucleotides resulting from the hybridization of the double-stranded nucleic acid of FIG. 3B to the immobilized capture oligonucleotides of FIG. 3C, followed by extension of the immobilized capture oligonucleotides, thereby generating an immobilized complement of each strand of the double-stranded nucleic acid of FIG. 3B.

### DETAILED DESCRIPTION

The present disclosure relates to methods for uniquely identifying sequencing reads from a double-stranded nucleic acid template. The disclosure further relates to sequencing adapters including landmark sequences (i.e., exogenous landmark sequences) that are uniquely associated with individual polynucleotide fragments.

### I. Definitions

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, N.Y. 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular forms “a,” “an” and “the” include plural referents unless the content clearly dictates otherwise. Thus, for example, reference to “a protein” includes a mixture of two or more proteins, and the like.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. As used herein, the terms “includes,” “including,” “includes,” “including,” “contains,” “containing,” “have,” “having,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter. Similarly, “comprise,” “comprises,” “comprising” “include,” “includes,” and “including” are interchangeable and not intended to be limiting.

Other than in the operating examples, or where otherwise indicated, all numbers expressing quantities of ingredients or reaction conditions used herein should be understood as modified in all instances by the term “about.” The term “about” when used to described aspects of the disclosure, in connection with percentages means ±1%, ±2%, ±3%, ±4%, ±5%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, or 6 to 9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain aspects, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application, and can be routinely determined by persons skilled in the art, without undue experimentation.

As used herein, the term “dNTP” refers to deoxynucleoside triphosphates. NTP refers to ribonucleotide triphosphates. The purine bases (Pu) include adenine (A), guanine (G) and derivatives and analogs thereof. The pyrimidine bases (Py) include cytosine (C), thymine (T), uracil (U) and derivatives and analogs thereof. Examples of such derivatives or analogs, by way of illustration and not limitation, are those which are modified with a reporter group, biotinylated, amine modified, radiolabeled, alkylated, and the like and also include phosphorothioate, phosphite, ring atom modified derivatives, and the like. The reporter group can be a fluorescent group such as fluorescein, a chemiluminescent group such as luminol, a terbium chelator such as N-(hydroxyethyl) ethylenediaminetriacetic acid that is capable of detection by delayed fluorescence, and the like.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an aspect, the other nucleic acid is a single-stranded nucleic acid. In some aspects, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some aspects, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other aspects, the stringency of the hybridization can be further altered by the addition or removal of components of the buffered solution.

As used herein, the terms “ligation,” “ligating,” and grammatical equivalents thereof are intended to mean to form a covalent bond or linkage between the termini of two or more nucleic acids, e.g., oligonucleotides and/or polynucleotides, typically in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide. Template driven ligation reactions are described in the following references: U.S. Pat. Nos. 4,883,750; 5,476,930; 5,593,826; and 5,871,921, incorporated herein by reference in their entireties. The term “ligation” also encompasses non-enzymatic formation of phosphodiester bonds, as well as the formation of non-phosphodiester covalent bonds between the ends of oligonucleotides, such as phosphorothioate bonds, disulfide bonds, and the like.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acid. For example, specific hybridization includes the hybridization of a primer or capture nucleic acid to a portion of a target nucleic acid (e.g., a template, or adapter portion of a template) that is substantially complementary to the primer or capture nucleic acid. In some aspects nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which comprises a double stranded portion of nucleic acid.

As may be used herein, the terms “nucleic acid,” “nucleic acid molecule,” “nucleic acid sequence,” “strand,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA (e.g., cDNA derived from RNA), a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some aspects, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some aspects, an oligonucleotide is a primer configured for extension by a polymerase when the primer is annealed completely or partially to a complementary nucleic acid template. A primer is often a single stranded nucleic acid. In certain aspects, a primer, or portion thereof, is substantially complementary to a portion of an adapter. In some aspects, a primer has a length of 200 nucleotides or less. In certain aspects, a primer has a length of 10 to 150 nucleotides, 15 to 150 nucleotides, 5 to 100 nucleotides, 5 to 50 nucleotides or 10 to 50 nucleotides. In some aspects, an oligonucleotide may be immobilized to a solid support.

The term “adapter” as used herein refers to any linear oligonucleotide that can be ligated to a nucleic acid molecule, thereby generating nucleic acid products that can be sequenced on a sequencing platform (e.g., an Illumina sequencing platform). In aspects, adapters include two reverse complementary oligonucleotides forming a double-stranded structure. In aspects, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In aspects, adapters include sequences that bind to sequencing primers. In aspects, adapters include sequences that bind to immobilized oligonucleotides (e.g., P7 and P5 sequences) or reverse complements thereof. In aspects, the adapter is substantially non-complementary to the 3′ end or the 5′ end of any target polynucleotide present in the sample. In aspects, the adapter can include a sequence that is substantially identical, or substantially complementary, to at least a portion of a primer, for example a universal primer. In aspects, the adapter can include an index sequence (also referred to as barcode or tag) to assist with downstream error correction, identification or sequencing.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some aspects, a primer has a length of 200 nucleotides or less. In certain aspects, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an aspect the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another aspect, the primer is an RNA primer. In aspects, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In aspects, a primer binding sequence is about or at least about 10, 15, 20, 25, 30, or more nucleotides in length. In aspects, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

As used herein, the term “nucleotide analogs” refers to synthetic analogs having modified nucleotide base portions, modified pentose portions, and/or modified phosphate portions, and, in the case of polynucleotides, modified internucleotide linkages, as generally described elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H+, NH4+, Na+, if such counterions are present. Exemplary modified nucleotide base portions include but are not limited to 5-methylcytosine (5mC); C-5-propynyl analogs, including but not limited to, C-5 propynyl-C and C-5 propynyl-U; 2,6-diaminopurine, also known as 2-amino adenine or 2-amino-dA); hypoxanthine, pseudouridine, 2-thiopyrimidine, isocytosine (isoC), 5-methyl isoC, and isoguanine (isoG; see, e.g., U.S. Pat. No. 5,432,272). Exemplary modified pentose portions include, but are not limited to, locked nucleic acid (LNA) analogs including without limitation Bz-A-LNA, 5-Me-Bz-C-LNA, dmf-G-LNA, and T-LNA (see, e.g., The Glen Report, 16(2):5, 2003; Koshkin et al., Tetrahedron 54:3607-30, 1998), and 2′-or 3′-modifications where the 2′-or 3′-position is hydrogen, hydroxy, alkoxy (e.g., methoxy, ethoxy, allyloxy, isopropoxy, butoxy, isobutoxy and phenoxy), azido, amino, alkylamino, fluoro, chloro, or bromo. Modified internucleotide linkages include phosphate analogs, analogs having achiral and uncharged intersubunit linkages (e.g., Sterchak, E. P. et al., Organic Chern., 52:4202, 1987), and uncharged morpholino-based polymers having achiral intersubunit linkages (see, e.g., U.S. Pat. No. 5,034,506). Some internucleotide linkage analogs include morpholidate, acetal, and polyamide-linked heterocycles.

As used herein, the term “degenerate bases” refers to a nucleotide that can perform the same function (i.e., base-pair) or yield the same output as a structurally different nucleotide. Examples of degenerate bases include, but are not limited to, inosine, the pyrimidine base 6H,8H-3,4-dihydropyrimido[4,5-c][1,2]oxazin-7-one (P), the purine base N⁶-methoxy-2,6-diaminopurine (K), and 5-nitroindole.

In the context of “polynucleotides,” the terms “variant” and “derivative” as used herein refer to a polynucleotide that comprises a nucleotide sequence of a polynucleotide or a fragment of a polynucleotide, which has been altered by the introduction of nucleotide substitutions, deletions or additions. A variant or a derivative of a polynucleotide can be a fusion polynucleotide which contains part of the nucleotide sequence of a polynucleotide. The term “variant” or “derivative” as used herein also refers to a polynucleotide or a fragment thereof, which has been chemically modified, e.g., by the covalent attachment of any type of molecule to the polynucleotide. For example, but not by way of limitation, a polynucleotide or a fragment thereof can be chemically modified, e.g., by acetylation, phosphorylation, methylation, etc. The variants or derivatives are modified in a manner that is different from naturally occurring or starting nucleotide or polynucleotide, either in the type or location of the molecules attached. Variants or derivatives further include deletion of one or more chemical groups which are naturally present on the nucleotide or polynucleotide. A variant or a derivative of a polynucleotide or a fragment of a polynucleotide can be chemically modified by chemical modifications using techniques known to those of skill in the art, including, but not limited to specific chemical cleavage, acetylation, formulation, etc. Further, a variant or a derivative of a polynucleotide or a fragment of a polynucleotide can contain one or more dNTPs or nucleotide analogs. A polynucleotide variant or derivative may possess a similar or identical function as a polynucleotide or a fragment of a polynucleotide described herein. A polynucleotide variant or derivative may possess an additional or different function compared with a polynucleotide or a fragment of a polynucleotide described herein.

As used herein, the term “double-stranded,” when used in reference to a nucleic acid molecule, means that substantially all of the nucleotides in the nucleic acid molecule are hydrogen bonded to a complementary nucleotide. A partially double stranded nucleic acid can have at least 10%, 25%, 50%, 60%, 70%, 80%, 90% or 95% of its nucleotides hydrogen bonded to a complementary nucleotide.

As used herein, the term “single-stranded,” when used in reference to a nucleic acid molecule, means that essentially none of the nucleotides in the nucleic acid molecule is hydrogen bonded to a complementary nucleotide.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g., a PCR product) or multiple copies of the nucleotide sequence (e.g., a concatemeric product of RCA). A first amplicon of a target nucleic acid can be a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

A nucleic acid can be amplified by a thermocycling method or by an isothermal amplification method. In some aspects, a rolling circle amplification method is used. In some aspects amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some aspects of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some aspects, solid phase amplification comprises a nucleic acid amplification reaction comprising only one species of oligonucleotide primer immobilized to a surface or substrate. In certain aspects solid phase amplification comprises a plurality of different immobilized oligonucleotide primer species. In some aspects, solid phase amplification may comprise a nucleic acid amplification reaction comprising one species of oligonucleotide primer immobilized on a solid surface and a second different oligonucleotide primer species in solution. Multiple different species of immobilized or solution based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

The number of template copies or amplicons that can be produced can be modulated by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. The number of copies of a nucleic acid template can be at least 1, 10, 100, 200, 500, 1000, 2000, 3000, 4000, 5000, 6000, 7000, 8000, 9000 and 10,000 copies, or a range that includes or is between any two of the foregoing numbers, and can be varied depending on the particular application.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “molecular barcode” (which may be referred to as a “tag”, a “barcode”, a “barcode sequence”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In aspects, a barcode is unique in a pool of barcodes that differ from one another in sequence, or is uniquely associated with a particular sample polynucleotide in a pool of sample polynucleotides. In aspects, every barcode in a pool of adapters is unique, such that sequencing reads comprising the barcode can be identified as originating from a single sample polynucleotide molecule on the basis of the barcode alone. In other aspects, individual barcode sequences may be used more than once, but adapters comprising the duplicate barcodes are associated with different sequences and/or in different combinations of barcoded adapters, such that sequence reads may still be uniquely distinguished as originating from a single sample polynucleotide molecule on the basis of a barcode and adjacent sequence information (e.g., sample polynucleotide sequence, and/or one or more adjacent barcodes). In aspects, barcodes are about or at least about 5, 6, 7, 8, 9, 10, 15, 20, 25, 30, 40, 50, 75 or more nucleotides in length. In aspects, barcodes are shorter than 20, 15, 10, 9, 8, 7, 6, or 5 nucleotides in length. In aspects, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In aspects, each barcode in a plurality of barcodes differs from every other barcode in the plurality by at least three nucleotide positions, such as at least 3, 4, 5, 6, 7, 8, 9, 10, or more nucleotide positions. In some aspects, substantially degenerate barcodes may be known as random barcodes. Additional methods of generating and using UMIs for sequencing applications are disclosed in, e.g., U.S. Pat. Nos. 10,844,428, 10,844,429, 11,371,094 and 11,421,238, and in U.S. Pat. Pubs. 2016/0326578, 2017/0211140, 2017/0247687, 2017/0306392, 2021/0238670, and 2021/0292836, each of which is incorporated herein by reference in its entirety.

As used herein, the term “landmark sequence” or “exogenous landmark sequence” refers to a nucleotide sequence includes one or more nucleotide analogs. The landmark sequence may also be referred to as a “landmark-inducing sequence (LIS)”. The landmark sequence can be used, for example, to generate a UMI through amplification of a landmark sequence-containing nucleic acid. For example, a landmark sequence may be included in an adapter that is ligated onto a template nucleic acid. A polymerase then extends a primer hybridized to the adapter-ligated template nucleic acid and generates a complement of the adapter-ligated template nucleic acid, generating a complement landmark sequence. The complement landmark sequence (also referred to herein as a landmark-locked sequence (LLS)) does not include the one or more nucleotide analogs of the parental exogenous landmark sequence, but instead includes nucleotides complementary to each of the nucleotide analogs of the parental exogenous landmark sequence. The complement landmark sequence facilitates downstream identification of the original template nucleic acid, and is capable of distinguishing an individual molecule in a large heterogeneous population of molecules. In aspects, the landmark sequence does not include a degenerate or random nucleotide sequence. In some aspects, the landmark sequence is a predetermined sequence.

As used herein, the term “endogenous landmark sequence” refers to a unique sub-sequence in a source DNA molecule. In some aspects, an endogenous landmark sequence is located at or near the ends of the source DNA molecule. One or more such unique end positions may alone or in conjunction with other information (e.g., an exogenous landmark sequence) uniquely identify a source DNA molecule. Depending on the number of distinct source DNA molecules and the number of nucleotides in the endogenous landmark sequence, one or more endogenous landmark sequences can uniquely identify source DNA molecules in a sample. In some aspects, a combination of two endogenous landmark sequence is required to identify a source DNA molecule. Such combinations may be extremely rare, possibly found only once in a sample. In some cases, one or more endogenous landmark sequences in combination with one or more exogenous landmark sequences may together uniquely identify a source DNA molecule.

As used herein, the term “DNA polymerase” and “nucleic acid polymerase” are used in accordance with their plain ordinary' meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA-or RNA-dependent RNA polymerase, and reverse transcriptase. In some cases, the DNA polymerase is 9° N polymerase or a variant thereof, E. coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-) A485L/Y409V, Phi29 DNA Polymerase (<p29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In aspects, the polymerase is a protein polymerase. Typically, a DNA polymerase adds nucleotides to the d'end of a DNA strand, one nucleotide at a time. In aspects, the DNA polymerase is a Pol I DNA polymerase, Pol II DNA polymerase, Pol III DNA polymerase, Pol IV DNA polymerase, Pol V DNA polymerase, Pol P DNA polymerase, Pol p DNA polymerase, Pol /. DNA polymerase, Pol o DNA polymerase, Pol a DNA polymerase, Pol 8 DNA polymerase, Pol 8 DNA polymerase, Pol r|DNA polymerase, Pol r DNA polymerase, Pol K DNA polymerase, Pol L, DNA polymerase, Pol y DNA polymerase, Pol 0 DNA polymerase, Pol n DNA polymerase, or a thermophilic nucleic acid polymerase (e.g. Therminator y, 9° N polymerase (exo-), Therminator II, Therminator III, or Therminator IX). In aspects, the DNA polymerase is a modified archaeal DNA polymerase. In aspects, the polymerase is a reverse transcriptase. For example, a polymerase catalyzes the addition of a next correct nucleotide to the 3′-OH group of the primer via a phosphodiester bond, thereby chemically incorporating the nucleotide into the primer.

As used herein, the term “template polynucleotide” or “template nucleic acid” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA (e.g., cDNA derived from RNA), RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that are to be ligated together will generally directly abut one another.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some aspects, a sequencing process described herein comprises contacting a template and an annealed primer with a suitable polymerase under conditions suitable for polymerase extension and/or sequencing. In aspects, sequencing generates one or more sequencing reads. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In aspects, the solid substrate is in the form of a chip, a bead, a well, a capillary tube, a slide, a wafer, a filter, a fiber, a porous media, or a column. In aspects, the solid substrate is gold, quartz, silica, plastic, silica, diamond, silver, metal, or polypropylene. In aspects, the solid substrate is porous.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, sequencing-by-ligation, and sequencing-by-binding.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In aspects, a sequencing read includes reading a barcode and a template nucleotide sequence. In aspects, a sequencing read includes reading a template nucleotide sequence. In aspects, a sequencing read includes reading a barcode and not a template nucleotide sequence. In aspects, a sequencing read includes a computationally derived string corresponding to the detected label. The sequence reads are optionally stored in an appropriate data structure for further evaluation. In aspects, a first sequencing reaction can generate a first sequencing read. The first sequencing read can provide the sequence of a first region of the polynucleotide fragment. In aspects, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some aspects, the nucleic acid template is optionally subjected to one or more additional rounds of sequencing using additional sequencing primers, thereby generating additional sequencing reads.

The term “paired end reads” refers to reads obtained from paired end sequencing that obtains one read from each end of a nucleic fragment. Paired end sequencing involves fragmenting DNA into sequences called inserts. In some protocols such as some used by Illumina, the reads from shorter inserts (e.g., on the order of tens to hundreds of bp) are referred to as short-insert paired end reads or simply paired end reads. In contrast, the reads from longer inserts (e.g., on the order of several thousands of bp) are referred to as mate pair reads. In this disclosure, short-insert paired end reads and long-insert mate pair reads may both be used and are not differentiated with regard to the process for determining sequences of DNA fragments. In some aspects, paired end reads include reads of about 20 bp to 1000 bp. In some aspects, paired end reads include reads of about 50 bp to 500 bp, about 80 bp to 150 bp, or about 100 bp.

“Synthetic” agents refer to non-naturally occurring agents, such as enzymes or nucleotides derived or constructed using human-made techniques. For example, s synthetic DNA polymerases refer to non-naturally occurring DNA polymerases such as those constructed by synthetic methods, mutated parent DNA polymerases such as truncated DNA polymerases and fusion DNA polymerases. Synthetic oligonucleotides such as adapter sequences or primers, include a human-designed sequence, typically configured to maximize yield and minimize off-target products, without introducing any biases. Examples of synthetic oligonucleotide sequences include P5, P7, or complementary sequences thereof (i.e., P5′ or P7′). The P5 and P7 primers are used on the surface of commercial flow cells for sequencing on various Illumina platforms, as described in U.S. Patent Publication No. 2011/0059865 A1.

The term “library” merely refers to a collection or plurality of template nucleic acid molecules which share common sequences at their 5′ ends (e.g., the first end) and common sequences at their 3′ ends (e.g., the second end). In aspects, a population of template nucleic acid molecules form a library.

As used herein, the term “substrate” or “solid support” refers to any material that can serve as a solid or semi-solid foundation for creation of features such as wells for the deposition of biopolymers, including nucleic acids, polypeptide and/or other polymers. A substrate as provided herein is modified, for example, or can be modified to accommodate attachment of biopolymers by a variety of methods well known to those skilled in the art. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above (e.g., cyclic olefin copolymers, polyacrylamide, cyclic olefin polymers, etc.), and multiwell microtiter plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon. In a particular aspect, a “substrate” or “solid support” as used herein includes, but is not limited to beads, a microarray, a plate, a multiwell plate, or a flowcell (e.g., a nonpatterned flowcell, or a pattered flowcell). The substrate can comprise a planar surface, or comprise a non-planar (e.g., convex or concave) surface. Those skilled in the art will know or understand that the composition and geometry of a substrate as provided herein can vary depending on the intended use and preferences of the user. In some aspects, the substrate may be patterned. For example, the substrate may be patterned with nanowells. Therefore, although planar substrates such as slides, chips or wafers are exemplified herein in reference to microarrays for illustration, given the teachings and guidance provided herein, those skilled in the art will understand that a wide variety of other substrates exemplified herein or well known in the art also can be used in the methods and/or compositions herein.

In a certain aspect, a “substrate” or “solid support” as disclosed herein may further comprises islands or clusters of immobilized capture agents or capture oligos. The islands or clusters can be generated on the surface of a substrate (e.g., a flowcell) by using bridge amplification. In such a case, the substrate comprises a plurality immobilized capture oligos on the surface of the substrate, which bind with complementary adapter regions presents on nearby primers or oligos to form bridge-like structures; these bridge-like structures are then extended using a polymerase enzyme, generating a double stranded molecule, that is then denatured to leave a single-stranded capture oligo anchored to the substrate. After multiple iterations of the foregoing process, islands or clusters of immobilized capture oligos are created. An example of the foregoing process that can be used with the methods and compositions disclosed herein can be found in WO 2022/015913 A1, which is incorporated herein by reference in-full. In a particular aspect, the nearby primers or oligos are attached to the substrate (e.g., a flowcell) by a selectively cleavable linker. Each island or cluster may be roughly circular or oval in shape. Each island or cluster may have an average diameter of 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1050 nm, 1100 nm, 1200 nm, or a range that includes or is in between any two of the forgoing diameters. In a further aspect, the surface of the substrate (e.g., a flowcell) comprises per 1 mm² of surface area 0.3, 0.4, 0.5, 0.6. 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, or 2.5 million clusters, or range including or between any two of the forgoing numbers. In a particular aspect, a “substrate” as disclosed herein comprises islands or clusters of immobilized capture oligos comprising adapter sequence(s), a spatial address sequence, an optional sequence primer site, and a capture moiety for a targeted analyte. In yet a further aspect, each cluster or island on the substrate (e.g., a flowcell) comprises capture oligos that have a unique spatial address sequence, so the x, y location of each cluster or island can be identified. In such a case, the x, y location of each cluster or island can be determined by decoding the spatial address sequence. Methods to decode the spatial address sequence include, but are not limited, the decoding-by-hybridization or the decoding-by-sequencing methods disclosed herein.

In some aspects, the substrate or solid support is an ordered substrate or solid support. An “ordered substrate” refers to an arrangement of different regions in or on an exposed layer of a substrate, where each region comprises features (e.g., nanowells) that have an assigned x, y spatial address, or an x, y spatial address that can be readily determined. An “ordered substrate” may have a specific pattern of features. In some aspects, the pattern can be a repeating arrangement of features and/or interstitial regions. In a certain aspect, the surface(s) of an “ordered substrate” can be patterned with spatial address sequences. Exemplary patterned substrate that can be used in the methods and compositions set forth herein are described in U.S. Ser. No. 13/661,524 or US Pat. App. Publ. No. 2012/0316086 A1, each of which is incorporated herein by reference. In a particular aspect, the features of an ordered substrate can comprise immobilized oligos, or islands or clusters of immobilized oligos. In such an aspect, the location of the islands or clusters of immobilized capture oligos can be readily be determined without having to decode the spatial address sequence of immobilized oligos. Accordingly, immobilized oligos having a unique spatial address sequence is optional for an “ordered substrate.” Examples of “ordered substrates” include, but are not limited to, patterned flowcells, beadchip arrays, and microarrays.

As used herein, the term “interstitial region” refers to an area in a substrate or on a surface that separates other areas of the substrate or surface. For example, an interstitial region can separate one feature of an array from another feature of the array. The two regions that are separated from each other can be discrete, lacking contact with each other. In another example, an interstitial region can separate a first portion of a feature from a second portion of a feature. The separation provided by an interstitial region can be partial or full separation. Interstitial regions will typically have a surface material that differs from the surface material of the features on the surface. For example, features of an array can have an amount or concentration of capture agents or capture oligos that exceeds the amount or concentration present at the interstitial regions. In some aspects, the capture agents or primers may not be present at the interstitial regions.

In some aspects, the substrate or solid support includes an array of wells or depressions in a surface. This may be fabricated as is generally known in the art using a variety of techniques, including, but not limited to, photolithography, stamping techniques, molding techniques and micro-etching techniques. As will be appreciated by those in the art, the technique used will depend on the composition and shape of the array substrate.

The features of a patterned substrate or an ordered substrate can be wells in an array of wells (e.g., microwells or nanowells) on glass, silicon, plastic or other suitable solid supports with patterned, covalently-linked gel such as poly(N-(5-azidoacetamidylpentyl)acrylamide-coacrylamide) (PAZAM, see, for example, U.S. Prov. Pat. App. Ser. No. 61/753,833, which is incorporated herein by reference). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However, in many aspects, the gel need not be covalently linked to the wells. For example, in some conditions silane free acrylamide (SFA, see, for example, U.S. Pat. App. Pub. No. 2011/0059865 A1, which is incorporated herein by reference) which is not covalently attached to any part of the structured substrate, can be used as the gel material.

In particular aspects, a patterned substrate or ordered substrate can be made by patterning a solid support material with wells (e.g., microwells or nanowells), coating the patterned support with a gel material (e.g., PAZAM, SFA or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of target nucleic acids (e.g., a fragmented human genome) can then be contacted with the polished substrate such that individual target nucleic acids will seed individual wells via interactions with primers attached to the gel material; however, the target nucleic acids will not occupy the interstitial regions due to absence or inactivity of the gel material. Amplification of the target nucleic acids will be confined to the wells since absence or inactivity of gel in the interstitial regions prevents outward migration of the growing nucleic acid colony. The process is conveniently manufacturable, being scalable and utilizing conventional micro- or nano-fabrication methods. A patterned substrate or ordered substrate can include, for example, wells etched into a slide or chip.

The pattern of the etchings and geometry of the wells can take on a variety of different shapes and sizes so long as such features are physically or functionally separable from each other. Particularly useful substrates having such structural features are patterned substrates that can select the size of solid support particles such as microspheres. An exemplary patterned substrate having these characteristics is the etched substrate used in connection with BeadArray technology (Illumina, Inc., San Diego, Calif). Further examples, are described in U.S. Pat. No. 6,770,441, which is incorporated herein by reference.

In some aspects, a substrate or solid support disclosed herein is a flowcell. The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference. A flowcell can be “a nonpatterned flowcell”, where the surface(s) of the flowcell comprises randomly or semi-randomly arranged features (e.g., areas comprising clusters or islands of oligos). Alternatively, the flowcell can be a “patterned flowcell,” where the flowcell comprises features (e.g., nanowells) at fixed locations across the surface(s) of the flowcell. The features of a “patterned flowcell” can further comprise immobilized oligos, or clusters or islands of immobilized oligos A “patterned flowcell” can be an “ordered substrate” in that the features of the patterned flowcell have an assigned x, y spatial address, or an x, y spatial address that can be readily determined.

As used herein, the term “immobilized” when used in reference to a nucleic acid is intended to mean direct or indirect attachment to a substrate or a feature of a substrate via covalent or non-covalent bond(s). In certain aspects, covalent attachment can be used, but all that is required is that the nucleic acids remain stationary or attached to a support under conditions in which it is intended to use the support, for example, in applications requiring nucleic acid amplification and/or sequencing. Oligonucleotides to be used as capture primers or amplification primers can be immobilized such that a 3′-end is available for enzymatic extension and at least a portion of the sequence is capable of hybridizing to a complementary sequence. Immobilization can occur via hybridization to a surface attached oligonucleotide, in which case the immobilized oligonucleotide or polynucleotide can be in the 3′-5′ orientation. Alternatively, immobilization of oligos can comprise use of a selectively cleavable linker. Examples of selectively cleavable linkers include, but are not limited to, biotin-based molecules (e.g., desthiobiotin molecule(s) (ddBio)), PC Linker, and a recognition site for a rare-cutter enzyme. Typically, the selectively cleavable linker can be cleaved by heating, competitive binding, pH change, chemical cleavage, enzymatic cleavage and/or photo-cleavage. Cleaving the selectively cleavable linker results in the release the nucleic acid, or a portion thereof, from the substrate or feature of the substrate.

Certain aspects may make use of an inert substrate or matrix (e.g., glass slides, polymer beads etc.) that has been functionalized, for example by application of a layer or coating of an intermediate material comprising reactive groups which permit covalent attachment to biomolecules, such as polynucleotides. Examples of such substrates include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such aspects, the biomolecules (e.g., polynucleotides) may be directly covalently attached to the intermediate material (e.g., the hydrogel) but the intermediate material may itself be non-covalently attached to the substrate or matrix (e.g., the glass substrate). The term “covalent attachment to a substrate” is to be interpreted accordingly as encompassing this type of arrangement.

Exemplary covalent linkages include, for example, those that result from the use of click chemistry techniques. Exemplary non-covalent linkages include, but are not limited to, non-specific interactions (e.g., hydrogen bonding, ionic bonding, van der Waals interactions etc.) or specific interactions (e.g., affinity interactions, receptor-ligand interactions, antibody epitope interactions, avidin-biotin interactions, streptavidin-biotin interactions, lectin carbohydrate interactions, etc.). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “plurality” is intended to mean a population of two or more different members. Pluralities can range in size from small, medium, large, to very large. The size of small plurality can range, for example, from a few members to tens of members. Medium sized pluralities can range, for example, from tens of members to about 100 members or hundreds of members. Large pluralities can range, for example, from about hundreds of members to about 1000 members, to thousands of members and up to tens of thousands of members. Very large pluralities can range, for example, from tens of thousands of members to about hundreds of thousands, a million, millions, tens of millions and up to or greater than hundreds of millions of members. Therefore, a plurality can range in size from two to well over one hundred million members as well as all sizes, as measured by the number of members, in between and greater than the above exemplary ranges. An exemplary number of features within a microarray includes a plurality of about 500,000 or more discrete features within 1.28 cm². Exemplary nucleic acid pluralities include, for example, populations of about 1×10⁵, 5×10⁵ and 1×10⁶ or more different nucleic acid species. Accordingly, the definition of the term is intended to include all integer values greater than two. An upper limit of a plurality can be set, for example, by the theoretical diversity of nucleotide sequences in a nucleic acid sample.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

Provided herein are methods and compositions for analyzing a sample (e.g., sequencing nucleic acids within a sample). A sample (e.g., a sample comprising nucleic acid) can be obtained from a suitable subject. A sample can be isolated or obtained directly from a subject or part thereof. In some aspects, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, parasite nucleic acid).

In some aspects, a sample comprises nucleic acid, or fragments thereof. A sample can comprise nucleic acids obtained from one or more subjects. In some aspects, a sample comprises nucleic acid obtained from a single subject. In some aspects, a sample comprises a mixture of nucleic acids. A mixture of nucleic acids can comprise two or more nucleic acid species having different nucleotide sequences, different fragment lengths, different origins (e.g., genomic origins, cell or tissue origins, subject origins, the like or combinations thereof), or combinations thereof. A sample may comprise synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some aspects, a subject is a mammal. In some aspects, a subject is a human subject. A subject can be a patient (e.g., a human patient). In some aspects, a subject is suspected of having a genetic variation or a disease or condition associated with a genetic variation.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

The recitation of a listing of elements in any definition of a variable herein includes definitions of that variable as any single element or combination (or subcombination) of listed elements. The recitation of an aspect herein includes that aspect as any single aspect or in combination with any other aspects or portions thereof.

All patents and publications mentioned in this specification are herein incorporated by reference to the same extent as if each independent patent and publication was specifically and individually indicated to be incorporated by reference.

### II. Methods

Some aspects of the present disclosure are directed to a method for amplifying a double-stranded nucleic acid molecule. In some aspects, the method comprises a first step a. of attaching adapters to both ends of a target double-stranded nucleic acid molecule, thereby generating a double-stranded nucleic acid molecule comprising a first strand adapter-target nucleic acid sequence and a second strand adapter-target nucleic acid sequence, wherein each adapter comprises a double-stranded region, and wherein each strand of the double-stranded region comprises a landmark sequence comprising a plurality of nucleotide analogs. The landmark sequence may also be referred to herein as a landmark-inducing sequence. In some aspects, the landmark sequence consists of an exogenous landmark sequence.

In some aspects, the method further comprises b. annealing a first primer to each of the first strand adapter-target nucleic acid sequence and the second strand adapter-target nucleic acid sequence and c. extending the annealed first primers with a polymerase, thereby generating a first double-stranded extension product comprising a complement of the first strand adapter-target nucleic acid, and a second double-stranded extension product comprising a complement of the second strand adapter-target nucleic acid sequence, wherein each of the complement of the first strand adapter-target nucleic acid sequence and complement of the second strand adapter-target nucleic acid sequence comprises a landmark sequence complement.

In some aspects, the method further comprises d. annealing a second primer to each of the complement of the first strand adapter-target nucleic acid sequence and the complement of the second strand adapter-target nucleic acid sequence and e. amplifying the complement of the first strand adapter-target nucleic acid sequence and the complement of the second strand adapter-target nucleic acid sequence with a polymerase, thereby generating a plurality of first amplification products and a plurality of second amplification products.

In an aspect is provided a method for amplifying a double-stranded nucleic acid molecule, the method comprising: a. attaching adapters to both ends of a target double-stranded nucleic acid molecule, thereby generating a double-stranded nucleic acid molecule comprising a first strand adapter-target nucleic acid sequence and a second strand adapter-target nucleic acid sequence, wherein each adapter comprises a double-stranded region, and wherein each strand of the double-stranded region comprises a landmark sequence comprising a plurality of nucleotide analogs; b. annealing a first primer to each of the first strand adapter-target nucleic acid sequence and the second strand adapter-target nucleic acid sequence; c. extending the annealed first primers with a polymerase, thereby generating a first double-stranded extension product comprising a complement of the first strand adapter-target nucleic acid, and a second double-stranded extension product comprising a complement of the second strand adapter-target nucleic acid sequence, wherein each of the complement of the first strand adapter-target nucleic acid sequence and complement of the second strand adapter-target nucleic acid sequence comprises a landmark sequence complement; d. annealing a second primer to each of the complement of the first strand adapter-target nucleic acid sequence and the complement of the second strand adapter-target nucleic acid sequence; and e. amplifying the complement of the first strand adapter-target nucleic acid sequence and the complement of the second strand adapter-target nucleic acid sequence with a polymerase, thereby generating a plurality of first amplification products and a plurality of second amplification products.

In some aspects, the first double-stranded extension product comprises random or semi-random complement bases to the degenerate base positions of the landmark sequence to create a unique or semi-unique landmark sequence complement. In some aspects, the second double-stranded extension product comprises random or semi-random complement bases to the degenerate base positions of the landmark sequence to create a unique or semi-unique landmark sequence complement.

In some aspects, the landmark sequence of the first strand adapter-target nucleic acid sequence and second strand adapter-target nucleic acid sequence are the same. In some aspects, the landmark sequence of the first strand adapter-target nucleic acid sequence and second strand adapter-target nucleic acid sequence are the same and the landmark sequence complements of the first double-stranded extension product and second double-stranded extension products are different.

In some aspects, the landmark sequence of the first strand adapter-target nucleic acid sequence and second strand adapter-target nucleic acid sequence are different. In some aspects, the landmark sequence of the first strand adapter-target nucleic acid sequence and second strand adapter-target nucleic acid sequence are different and the landmark sequence complements of the first double-stranded extension product and second double-stranded extension products are different.

In some aspects, the first strand adapter-target nucleic acid sequence and second strand adapter-target nucleic acid sequence are removed after step c and before step d. In some aspects, the first strand adapter-target nucleic acid sequence and second strand adapter-target nucleic acid sequences are removed by nuclease treatment. In some aspects, the first strand adapter-target nucleic acid sequence and second strand adapter-target nucleic acid sequences are removed by chromatography. In some aspects, the chromatography comprises size exclusion chromatography or immobilized metal affinity chromatography (IMAC). In some aspects, the first strand adapter-target nucleic acid sequence and second strand adapter-target nucleic acid sequences are removed by ultrafiltration. In some aspects, the ultrafiltration comprises membrane filtration.

In some aspects, the first strand adapter-target nucleic acid sequence and second strand adapter-target nucleic acid sequences may incorporate sequence components that prevent them from participating in subsequent amplification events after a first round of amplification or extension. In some aspects, the first strand adapter-target nucleic acid sequence and second strand adapter-target nucleic acid sequences may include sequence elements such as uracil containing bases that can prevent their amplification by certain polymerases e.g., that are present in later rounds of amplification.

In some aspects, unincorporated first primers (i.e., first primers which were not extended by the polymerase in step c.) are removed after step c. and before step d. In some aspects, the unincorporated first primers are removed by single-stranded nuclease treatment. In some aspects, following single-stranded nuclease treatment, the first double-stranded extension product and the second double-stranded extension product of step c are purified. In some aspects, the single-stranded nuclease is S1 nuclease, P1 nuclease, or exonuclease VII.

In some aspects, the unincorporated first primers are removed by chromatography. In some aspects, the chromatography comprises size exclusion chromatography or immobilized metal affinity chromatography (IMAC). Examples of IMAC removal of unincorporated primers using, e.g., Cu²⁺ iminodiacetic acid agarose are described in Kanakaraj I et al. PLOS One. 2011; 6(1): e14512. In some aspects, the unincorpoated primers are removed by ultrafiltration. In some aspects, the ultrafiltration comprises membrane filtration.

In some aspects, the first primer comprises an affinity tag at the 5′ end. In some aspects, the affinity tag is a biotin moiety. In some aspects, the first and second double-stranded extension products of step c each comprise the affinity tag (e.g., each comprise a biotin moiety at a 5′ end). In some aspects, after step c, the first double-stranded extension product comprising the affinity tag and the second double-stranded extension product comprising the affinity tag are contacted with a solid support comprising an immobilized capture agent (e.g., an avidin or strepavidin moiety), wherein the capture agent binds to the affinity tag of each double-stranded extension product. In some aspects, the immobilized capture agent comprises an avidin moiety. In some aspects, the immobilized capture agent comprises a strepavidin moiety. In some aspects, after binding of each double-stranded extension product to the solid support, the double-stranded nucleic acids are denatured, and the first strand adapter-target nucleic acid sequence and second strand nucleic acid sequence (i.e., the strands that are not bound to the solid support) are removed.

In some aspects, the adapters comprise an affinity tag (e.g., a biotin moiety) at a 5′ end (i.e., at a 5′ end of one of the polynucleotide strands of the adapter). In some aspects, the method further comprises, after step a, immobilizing the double-stranded nucleic acid molecule of step a to a solid support comprising a capture agent (e.g., an avidin or strepavidin moiety). In some aspects, step b comprises annealing a first primer to each of the immobilized first strand adapter-target nucleic acid sequence and the immobilized second strand adapter-target nucleic acid sequence. In some aspects, after step c, the double-stranded nucleic acids are denatured, and the complement of the first strand adapter-target nucleic acid sequence and the complement of the second strand nucleic acid sequence (i.e., the strands of the double-stranded nucleic acids that are not bound to the solid support) are eluted and subjected to steps d and e.

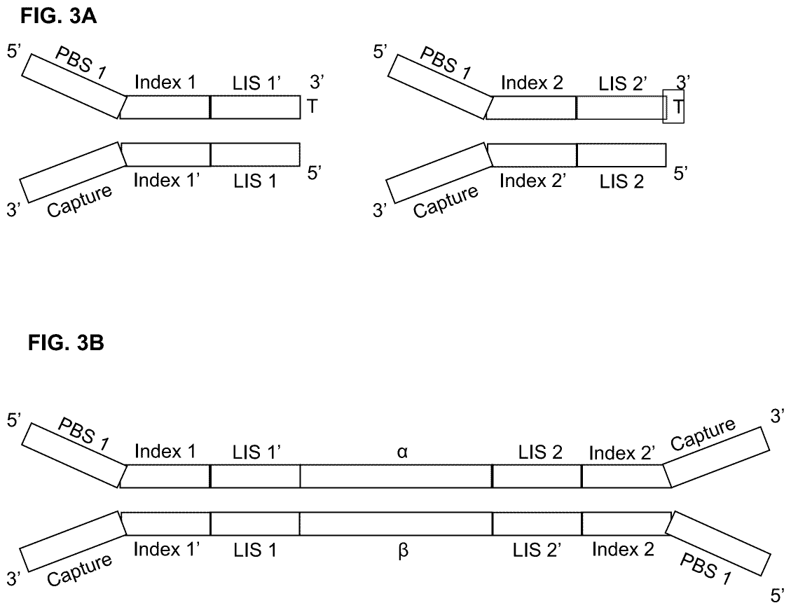

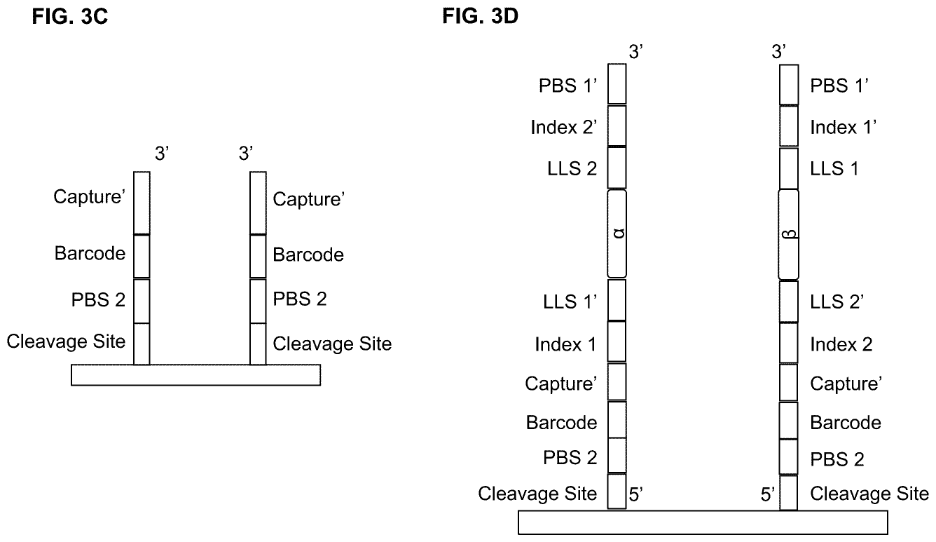

In some aspects, the adapters are Y-shaped adapters comprising a single-stranded 5′ arm, a single-stranded 3′ arm, and a double-stranded region comprising the landmark sequence. In some aspects, the 3′ arm comprises a capture sequence (see, e.g., FIG. 3A). In some aspects, the 3′ end of the single-stranded region is blocked (e.g., the 3′ end does not comprise a 3′-OH) to prevent extension. In some aspects, the 5′ arm comprises a primer binding sequence (e.g, a first primer binding sequence). In some aspects, the double-stranded region of the Y-shaped adapter further comprises an index sequence. In some aspects, the adapters comprise a first plurality of Y-shaped adapters and a second plurality of Y-shaped adapters. In some aspects, the double-stranded region of the first plurality of Y-shaped adapters comprise a first index sequence and the second plurality of Y-shaped adapters comprise a second index sequence. In some aspects, the indeces are used for sample identification (i.e., identification of individual samples from a plurality of samples). In some aspects, the indeces are used for strand identification (i.e., identification and/or tracking of each strand of the double-stranded nucleic acid). In some aspects, the indeces are used for error correction. Exemplary methods for using indeces for error correction are described in, e.g., U.S. Pat. Nos. 10,844,428 and 10,844,429, each of which is incorporated herein by reference.

In some aspects, the double-stranded region of the first plurality of Y-shaped adapters comprise a first landmark sequence and the second plurality of Y-shaped adapters comprise a second landmark sequence. In some aspects, step a comprises attaching the Y-shaped adapters to both ends of the target double-stranded nucleic acid molecule. In some aspects, step a comprises attaching the first plurality of Y-shaped adapters and the second plurality of Y-shaped adatpers to each end of the target double-stranded nucleic acid molecule, respectively, thereby generating a double-stranded nucleic acid molecule comprising a Y-shaped adapter of the first plurality at one end and a Y-shaped adapter of the second plurality at the second end (see, e.g., FIG. 3B). In some aspects, the first strand of the double-stranded nucleic acid molecule comprises, from 5′ to 3′, a first primer binding sequence, a first index sequence, a first landmark sequence complement, a target nucleic acid sequence, a second landmark sequence, a second index sequence complement, and a capture sequence. In some aspects, the second strand of the double-stranded nucleic acid molecule (i.e., the strand annealed to the first strand of the double-stranded nucleic acid molecule) comprises, from 5′ to 3′, a first primer binding sequence, a second index sequence, a second landmark sequence complement, a target nucleic acid sequence complement, a first landmark sequence, a first index sequence complement, and the capture sequence.

In some aspects, the method further comprises hybridizing the double-stranded nucleic acid molecule comprising the Y-shaped adapters to a solid support comprising immobilized capture oligonucleotides, thereby forming double-stranded nucleic acid molecules complexes. In some aspects, the immobilized capture oligonucleotides comprise, from 5′ to 3′, a cleavage site, a primer binding sequence (e.g., a second primer binding sequence) and a capture sequence complement (i.e., a sequence complementary to the capture sequence of the Y-shape adapters). In some aspects, the immobilized capture oligonucleotides further comprises a barcode sequence (e.g., a bead index sequence or a cellular barcode sequence). In some aspects, the immobilized capture oligonucleotides comprise, from 5′ to 3′, the cleavage site, a second primer binding sequence, the barcode sequence, and the capture sequence complement (see, e.g., FIG. 3C).

In some aspects, the method further comprises extending the immobilized capture oligonucleotides of the double-stranded nucleic acid molecule complexes, thereby generating a first immobilized extended capture oligonucleotide and a second immobilized extended capture oligonucleotide (see, e.g., FIG. 3D). In some aspects, the first immobilized extended capture oligonucleotide comprises, from 5′ to 3′, the cleavage site, the second primer binding sequence, the capture sequence complement, the first index sequence, the first landmark sequence complement, the second landmark sequence, the second index sequence complement, and a first primer binding sequence complement. In some aspects, the second immobilized extended capture oligonucleotide comprises, from 5′ to 3′, the cleavage site, the second primer binding sequence, the capture sequence complement, the second index sequence, the second landmark sequence complement, the first landmark sequence, the first index sequence complement, and a first primer binding sequence complement. In some aspects, the first immobilized extended capture oligonucleotide and the second immobilized extended capture oligonucleotide each comprise a barcode sequence.

In some aspects, following the extending, unbound nucleic acids (e.g., unbound double-stranded nucleic acid molecules) are removed from the solid support (e.g., removed by denaturation and/or washing). In some aspects, the method further comprises contacting the cleavage site of the first and second immobilized extended capture oligonucleotides with a cleaving agent, thereby releasing the first and second immobilized extended capture oligonucleotides from the solid support. In some aspects, the cleaving agent is an enzymatic cleaving agent or a chemical cleaving agent. In some aspects, the enzymatic cleaving agent is a restriction endonuclease. In some aspects, the method further comprises amplifying the released first and second immobilized extended capture oligonucleotides. In some aspects, the amplification comprises hybridizing a first primer to the first primer binding sequence complement and a second primer to the second primer binding sequence, and extending the first and second primers with a polymerase. In some aspects, the method further comprises sequencing the amplified first and second immobilized extended captures oligonucleotides.

In some aspects, the plurality of first amplifications products is distinguishable from the plurality of second amplification products.

In some aspects, the plurality of first amplification products comprises a sequence of the first strand of the target double-stranded nucleic acid molecule, and complement thereof, and wherein the plurality of second amplification products comprises the second strand of the target double-stranded nucleic acid molecule, and complement thereof.

In some aspects, the landmark sequence complement does not comprise nucleotide analogs.

In some aspects, each landmark sequence comprises one or more nucleotide analogs. In some aspects, each landmark sequence comprises 3 or more nucleotide analogs. In some aspects, each landmark sequence comprises 6 or more nucleotide analogs. In some aspects, each landmark sequence comprises 1, 2, 3, 4, 5, 6, or more nucleotide analogs.

In some aspects, the nucleotide analogs (i.e., the nucleotide analogs of the landmark sequence) are not adjacent to each other. In some aspects, each of the nucleotide analogs is separated by at least 2 nucleotides. In some aspects, each of the nucleotide analogs is separated by at least 3 nucleotides. In some aspects, each of the nucleotide analogs is separated by at least 4 nucleotides. In some aspects, each of the nucleotide analogs is separated by at least 5 nucleotides.

In some aspects, the nucleotides analogs comprise one or more degenerate bases. In some aspects, the one or more degenerate bases comprise inosine, the pyrimidine base 6H,8H-3,4-dihydropyrimido[4,5-c][1,2]oxazin-7-one (P), the purine base N⁶-methoxy-2,6-diaminopurine (K), 5-nitroindole, or any combination thereof. In some aspects, when the one or more degenerate bases comprise inosine, step c of the method is performed in the presence of a nucleotide mixture comprising uracil, adenine, thymine, cytosine, and guanine. In some aspects, the concentration of cytosine is reduced in comparison to the other nucleotides of the nucleotide mixture. In some aspects, the concentration of each nucleotide in the nucleotide mixture is adjusted to promote alternate base pairing with the one or more degenerate bases. In some aspects, the concentration of each nucleotide in the nucleotide mixture is adjusted to promote alternate base pairing with inosine (see, e.g., Licht K et al. Nucleic Acids Res. 2019; 47(1):3-14). Additional disclosure and methods of using degenerate bases is described in, e.g., Loakes D and Brown D M. Nucleic Acids Res. 1994; 22(2):4039-43; Lin P K and Brown D M. Nucleic Acids Res. 1989; 17(24): 10373-83; Lin P K and Brown D M. Nucleic Acids Res. 1992; 20:5149-52; Liu H and Nichols R. Biotechniques. 1994; 16:24-26, and U.S. Pat. No. 11,371,094, each of which is incorporated herein by reference in its entirety.

In some aspects, the first strand of the adapter comprises a first landmark sequence and wherein the second strand of the adapter comprises a second landmark sequence. In some aspects, the first landmark sequence and the second landmark sequence are substantially complementary. In some aspects, the first landmark sequence consists of a first exogenous landmark sequence and the second landmark sequence consists of a second exogenous landmark sequence, wherein each of the exogenous landmark sequences comprises one or more nucleotide analogs. In some aspects, the first landmark sequence is adjacent to a first endogenous landmark sequence and the second landmark sequence is adjacent to a second endogenous landmark sequence, wherein each endogenous landmark sequence corresponds to a breakpoint in the target double-stranded nucleic acid molecule.

Nucleic acid fragments may include sequences on each end that may be used as endogenous landmark sequences in some aspects of the methods described herein. For example, the endogenous landmark sequences, when used alone or combined with the exogenous landmark sequences of an adapter to be ligated to the fragment, may uniquely identify the fragment. Landmark sequences are uniquely associated with a single DNA fragment in a sample including a source polynucleotide and its complementary strand. An exogenous landmark sequence is a sequence of an oligonucleotide linked to the source polynucleotide, its complementary strand, or a polynucleotide derived from the source polynucleotide. An endogenous landmark sequence is a sequence of an oligonucleotide within the source polynucleotide, its complementary strand, or a polynucleotide derived from the source polynucleotide. Within this scheme, one may also refer to the exogenous landmark sequence as a physical UMI, and the endogenous landmark sequence as a virtual UMI.

Endogenous landmark sequences refer to two complementary sequences at the same genomic site. Endogenous landmark sequences can be used to help identify reads originating from one or both strands of the single DNA source fragment. With the reads so identified, they can be collapsed to obtain a consensus sequence.

Endogenous landmark sequences that are defined at, or with respect to, the end positions of source DNA molecules can uniquely or nearly uniquely define individual source DNA molecules when the locations of the end positions are generally random as with some fragmentation procedures and with naturally occurring cfDNA. When the sample contains relatively few source DNA molecules, the endogenous landmark sequences can themselves uniquely identify individual source DNA molecules. Using a combination of two endogenous landmark sequences, each associated with a different end of a source DNA molecule, increases the likelihood that endogenous landmark sequences alone can uniquely identify source DNA molecules. Of course, even in situations where one or two endogenous landmark sequences cannot alone uniquely identify source DNA molecules, the combination of such endogenous landmark sequences with one or more exogenous landmark sequences may succeed.

If two reads are derived from the same DNA fragment, two subsequences having the same base pairs will also have the same relative location in the reads. On the contrary, if two reads are derived from two different DNA fragments, it is unlikely that two subsequences having the same base pairs have the exact same relative location in the reads. Therefore, if two or more subsequences from two or more reads have the same base pairs and the same relative location on the two or more reads, it can be inferred that the two or more reads are derived from the same fragment.

In some aspects, subsequences at or near the ends of a DNA fragment are used as endogenous landmark sequences. This design choice has some practical advantages. First, the relative locations of these subsequences on the reads are easily ascertained, as they are at or near the beginning of the reads and the system need not use an offset to find the endogenous landmark sequence. Furthermore, since the base pairs at the ends of the fragments are first sequenced, those base pairs are available even if the reads are relatively short. Moreover, base pairs determined earlier in a long read have lower sequencing error rate than those determined later. In other implementations, however, subsequences located away from the ends of the reads can be used as endogenous landmark sequences, but their relative positions on the reads may need to be ascertained to infer that the reads are obtained from the same fragment.

One or more subsequences in a read may be used as endogenous landmark sequences. In some implementations, two subsequences, each tracked from a different end of the source DNA molecule, are used as endogenous landmark sequences. In various aspects, endogenous landmark sequences are about 24 base pairs or shorter, about 20 base pairs or shorter, about 15 base pairs or shorter, about 10 base pairs or shorter, about 9 base pairs or shorter, about 8 base pairs or shorter, about 7 base pairs or shorter, or about 6 base pairs or shorter. In some aspects, endogenous landmark sequences are about 6 to 10 base pairs. In other aspects, endogenous landmark sequences are about 6 to 24 base pairs.

In some aspects, the plurality of first amplification products is related to the plurality of second amplification products by the first exogenous landmark sequence, the first endogenous landmark sequence, or a combination thereof. In some aspects, the plurality of first amplification products is related to the plurality of second amplification products by the second exogenous landmark sequence, the second endogenous landmark sequence, or a combination thereof. In some aspects, the plurality of first amplification products is related to the plurality of second amplification products by the first exogenous landmark sequence, the first endogenous landmark sequence, the second exogenous landmark sequence, the second endogenous landmark sequence, or a combination thereof.

In some aspects, the first primer comprises a sequence complementary to a region of each of the first strand adapter-target nucleic acid sequence and the second strand adapter-target nucleic acid sequence 3′ of the landmark sequence.

In some aspects, the first primer comprises a first index sequence and one or more primer binding sequences.

In some aspects, the second primer comprises a second index sequence and one or more primer binding sequences.

In some aspects, each adapter comprises a single-stranded 5′ arm and a single-stranded 3′ arm. In some aspects, the first primer is complementary to a portion of the single-stranded 3′ arm. In some aspects, the second primer is complementary to a portion of the complement of the single-stranded 5′ arm.

In some aspects, each adapter further comprises a random sequence. In some aspects, the random sequence is between 2 to 8 nucleotides in length. In some aspects, the random sequence is 2, 3, 4, 5, 6, 7, or 8 nucleotides in length.

In some aspects, the adapter attached to the first strand adapter-target nucleic acid sequence and the adapter attached to the second strand adapter-target nucleic acid sequence comprise different landmark sequences.

In some aspects, the adapter is between 25 and 80 nucleotides in length. In some aspects, the adapter is greater than 80 nucleotides in length. In some aspects, the adapter is about 25, about 40, about 50, about 60, about 70, or about 80 nucleotides in length.

In some aspects, the amplifying of step (e) comprises PCR amplification.

In some aspects, prior to step (b), the method comprises denaturing the double-stranded nucleic acid molecule. In some aspects, the denaturing comprises chemical denaturation, thermal denaturation, or both chemical and thermal denaturation.

In some aspects, prior to step (d), the method comprises denaturing the first and second double-stranded extension products. In some aspects, the denaturing comprises chemical denaturation, thermal denaturation, or both chemical and thermal denaturation.

In some aspects, the method further comprises step (f) sequencing the plurality of first amplification products, thereby generating a plurality of first sequence reads, and sequencing the plurality of second amplification products, thereby generating a plurality of second sequence reads. In some aspects, the method further comprises step (g) comparing at least one sequence of the plurality of first sequence reads with at least one sequence obtained from the plurality of second sequence reads, thereby generating a consensus sequence of the double-stranded target nucleic acid molecule. In some aspects, generating the consensus sequence comprises grouping the at least one sequence of the plurality of first sequence reads with at least one sequence obtained from the plurality of second sequence reads is based at least on the landmark sequence, or complement thereof.

In some aspects, multiple sequence reads having the same landmark sequences are collapsed to obtain one or more consensus sequences, which are then used to determine the sequence of a source DNA molecule. Multiple distinct reads may be generated from distinct instances of the same source DNA molecule, and these reads may be compared to produce a consensus sequence as described herein. The instances may be generated by amplifying a source DNA molecule prior to sequencing, such that distinct sequencing operations are performed on distinct amplification products, each sharing the source DNA molecule's sequence. Of course, amplification may introduce errors such that the sequences of the distinct amplification products have differences. In the context of some sequencing technologies such as Illumina's sequencing-by-synthesis, a source DNA molecule or an amplification product thereof forms a cluster of DNA molecules linked to a region of a flow cell. The molecules of the cluster collectively provide a read. Typically, at least two reads are required to provide a consensus sequence. Sequencing depths of 100, 1000, and 10,000 are examples of sequencing depths useful in the disclosed aspects for creating consensus reads for low allele frequencies (e.g., about 1% or less).

In some aspects, nucleotides that are consistent across 100% of the reads sharing a landmark sequence or combination of landmark sequences are included in the consensus sequence. In other aspects, consensus criterion can be lower than 100%. For instance, a 90% consensus criterion may be used, which means that base pairs that exist in 90% or more of the reads in the group are included in the consensus sequence. In various aspects, the consensus criterion may be set at about 30%, about 40%, about 50%, about 60%, about 70%, about 80%, about 90%, about 95%, or about 100%.

Multiple techniques may be used to collapse reads that include multiple landmark sequences. In some implementations, reads sharing a common exogenous landmark sequence may be collapsed to obtain a consensus sequence. In some implementations, reads sharing a common landmark sequence complement may be collapsed to obtain a consensus sequence. In some aspects, an exogenous landmark sequence may not be unique enough by itself to identify a particular source molecule. In such a case, an exogenous landmark sequence may be combined with an endogenous landmark sequence to provide an index of the source molecule.

In one example, an exogenous landmark sequence 1 produces sequencing reads having exogenous landmark sequence 1. If all adapters used in a workflow have different exogenous landmark sequences, all reads having exogenous landmark sequence 1 at the adapter region are likely derived from the same strand of the DNA fragment. Similarly, an exogenous landmark sequence 2 would produce reads having exogenous landmark sequence 2, all of which are derived from the same complementary strand of the DNA fragment. It is therefore useful to collapse all reads including exogenous landmark sequence 1 to obtain one consensus sequence, and to collapse all reads including exogenous landmark sequence 2 to obtain another consensus sequence. Because all reads in a group are derived from the same source polynucleotide in a sample, base pairs included in the consensus sequence likely reflect the true sequence of the source polynucleotide, while a base pair excluded from the consensus sequence likely reflects a variation or error introduced in the workflow.

In addition, the endogenous landmark sequences at each end of a double-stranded nucleic acid fragment can provide information to determine that reads including one or both endogenous landmark sequences are derived from the same source DNA fragment. Because endogenous landmark sequences are internal to the source DNA fragments, the exploitation of the endogenous landmark sequences do not add overhead to preparation or sequencing in practice. After obtaining the sequences of the exogenous landmark sequences from reads, one or more sub-sequences in the reads may be determined as endogenous landmark sequences. If the endogenous landmark sequences include sufficient base pairs and have the same relative location on reads, they may uniquely identify the reads as having been derived from the source DNA fragment. Therefore, reads having one or both endogenous landmark sequences from the double-stranded nucleic acid fragment ends may be collapsed to obtain a consensus sequence. The combination of endogenous landmark sequences and exogenous landmark sequences can provide information to guide a second-level collapsing when only one exogenous landmark sequence is assigned to a first level consensus sequence of each strand. However, in some implementations, this second level collapsing using endogenous landmark sequences may be difficult if there are over-abundant input DNA molecules or fragmentation is not randomized.

In alternative aspects, reads having two exogenous landmark sequences on both ends may be collapsed in a second-level collapsing based on a combination of the exogenous landmark sequences and the endogenous landmark sequences. This is especially helpful when the exogenous landmark sequences are too short to uniquely identify source DNA fragments without using the endogenous landmark sequences. In these aspects, second level collapsing can be implemented, with physical duplex UMIs, by collapsing reads with dual exogenous landmark sequences and dual endogenous landmark sequences and consensus reads from the same DNA molecule, thereby obtaining a consensus sequence including nucleotides consistent among all of the reads.

Using the landmark sequence and collapsing scheme described herein, various aspects can suppress different sources of error affecting the determined sequence of a fragment even if the fragment includes alleles with very low allele frequencies. Reads sharing the same landmark sequences (exogenous and/or endogenous) are grouped together. By collapsing the grouped reads, variants (SNV and small indels) due to PCR, library preparation, clustering, and sequencing errors can be eliminated.

Regardless of the specific sequencing platform and protocol, at least a portion of the nucleic acids contained in the sample are sequenced to generate tens of thousands, hundreds of thousands, or millions of sequence reads, e.g., 100 bp reads. In some aspects, the sequence reads comprise about 20 bp, about 25 bp, about 30 bp, about 35 bp, about 36 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, about 500 bp, about 800 bp, about 1000 bp, or about 2000 bp.

In some aspects, reads are aligned to a reference genome, e.g., hg19. In other aspects, reads are aligned to a portion of a reference genome, e.g., a chromosome or a chromosome segment. The reads that are uniquely mapped to the reference genome are known as sequence tags. In one aspects, at least about 3×10⁶ qualified sequence tags, at least about 5×10⁶ qualified sequence tags, at least about 8×10⁶ qualified sequence tags, at least about 10×10⁶ qualified sequence tags, at least about 15×10⁶ qualified sequence tags, at least about 20×10⁶ qualified sequence tags, at least about 30×10⁶ qualified sequence tags, at least about 40×10⁶ qualified sequence tags, or at least about 50×10⁶ qualified sequence tags are obtained from reads that map uniquely to a reference genome.

In some aspects, prior to step (a), the adapter comprises a 3′ overhang.

In some aspects, prior to step (a), the adapter is blunt-ended.

In some aspects, the double-stranded nucleic acid molecule comprises DNA, RNA, or both DNA and RNA. In some aspects, the double-stranded nucleic acid molecule consists of DNA. In some aspects, the DNA is genomic DNA or cDNA derived from RNA.

In some aspects, the double-stranded nucleic acid molecule is isolated from a biological sample. In some aspects, the biological sample is a single cell or a tissue sample.

Samples that are used for determining DNA fragment sequence can include samples taken from any cell, fluid, tissue, or organ including nucleic acids in which sequences of interest are to be determined. In some aspects involving diagnosis of cancers, circulating tumor DNA may be obtained from a subject's bodily fluid, e.g. blood or plasma. In some aspects involving diagnosis of fetus, it is advantageous to obtain cell-free nucleic acids, e.g., cell-free DNA (cfDNA), from maternal body fluid. Cell-free nucleic acids, including cell-free DNA, can be obtained by various methods known in the art from biological samples including but not limited to plasma, serum, and urine (see, e.g., Fan et al., Proc Natl Acad Sci 105:16266-16271 [2008]; Koide et al., Prenatal Diagnosis 25:604-607 [2005]; Chen et al., Nature Med. 2: 1033-1035 [1996]; Lo et al., Lancet 350: 485-487 [1997]; Botezatu et al., Clin Chem. 46: 1078-1084, 2000; and Su et al., J Mol. Diagn. 6: 101-107 [2004]).

In various aspects the nucleic acids (e.g., DNA or RNA) present in the sample can be enriched specifically or non-specifically prior to use (e.g., prior to preparing a sequencing library). Non-specific enrichment of sample DNA refers to the whole genome amplification of the genomic DNA fragments of the sample that can be used to increase the level of the sample DNA prior to preparing a cfDNA sequencing library. Methods for whole genome amplification are known in the art. Degenerate oligonucleotide-primed PCR (DOP), primer extension PCR technique (PEP) and multiple displacement amplification (MDA) are examples of whole genome amplification methods. In some aspects, the sample is un-enriched for DNA.

The sample including the nucleic acids to which the methods described herein are applied typically include a biological sample (“test sample”) as described above. In some aspects, the nucleic acids to be sequenced are purified or isolated by any of a number of well-known methods.

Accordingly, in certain aspects, the sample includes or consists essentially of a purified or isolated polynucleotide, or it can include samples such as a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some aspects, the sample is a sample that is easily obtainable by non-invasive procedures, e.g., blood, plasma, serum, sweat, tears, sputum, urine, stool, sputum, ear flow, saliva or feces. In certain aspects, the sample is a peripheral blood sample, or the plasma and/or serum fractions of a peripheral blood sample. In other aspects, the biological sample is a swab or smear, a biopsy specimen, or a cell culture. In another aspect, the sample is a mixture of two or more biological samples, e.g., a biological sample can include two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain aspects, samples can be obtained from sources, including, but not limited to, samples from different individuals, samples from different developmental stages of the same or different individuals, samples from different diseased individuals (e.g., individuals suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative, but non-limiting example, the sample is a maternal sample that is obtained from a pregnant female, for example a pregnant woman. In this instance, the sample can be analyzed using the methods described herein to provide a prenatal diagnosis of potential chromosomal abnormalities in the fetus. The maternal sample can be a tissue sample, a biological fluid sample, or a cell sample. A biological fluid includes, as non-limiting examples, blood, plasma, serum, sweat, tears, sputum, urine, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, and leukophoresis samples.

In certain aspects, samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

Methods of isolating nucleic acids from biological sources are well known and will differ depending upon the nature of the source. One of skill in the art can readily isolate nucleic acids from a source as needed for the method described herein. In some instances, it can be advantageous to fragment the nucleic acid molecules in the nucleic acid sample. Fragmentation can be random, or it can be specific, as achieved, for example, using restriction endonuclease digestion. Methods for random fragmentation are well known in the art, and include, for example, limited DNAse digestion, alkali treatment and physical shearing.

In various aspects, sequencing may be performed on various sequencing platforms that require preparation of a sequencing library. The preparation typically involves fragmenting the DNA (sonication, nebulization or shearing), followed by DNA repair and end polishing (blunt end or A overhang), and platform-specific adapter ligation. In one aspects, the methods described herein can utilize next generation sequencing technologies (NGS), that allow multiple samples to be sequenced individually as genomic molecules (i.e., singleplex sequencing) or as pooled samples comprising indexed genomic molecules (e.g., multiplex sequencing) on a single sequencing run. These methods can generate up to several billion reads of DNA sequences. In various aspects the sequences of genomic nucleic acids, and/or of indexed genomic nucleic acids can be determined using, for example, the Next Generation Sequencing Technologies (NGS) described herein. In various aspects, analysis of the massive amount of sequence data obtained using NGS can be performed using one or more processors as described herein.

In various aspects the use of such sequencing technologies does not involve the preparation of sequencing libraries.

However, in certain aspects the sequencing methods contemplated herein involve the preparation of sequencing libraries. In one illustrative approach, sequencing library preparation involves the production of a random collection of adapter-modified DNA fragments (e.g., polynucleotides) that are ready to be sequenced. Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain aspects, the polynucleotides may originated in single-stranded form (e.g., ssDNA, RNA, etc.) and have been converted to dsDNA form. By way of illustration, in certain aspects, single stranded mRNA molecules may be copied into double-stranded cDNAs suitable for use in preparing a sequencing library. The precise sequence of the primary polynucleotide molecules is generally not material to the method of library preparation, and may be known or unknown. In one aspect, the polynucleotide molecules are DNA molecules. More particularly, in certain aspects, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as noncoding regulatory sequences such as promoter and enhancer sequences. In certain aspects, the primary polynucleotide molecules comprise human genomic DNA molecules, e.g., cfDNA molecules present in peripheral blood of a pregnant subject.

Preparation of sequencing libraries for some NGS sequencing platforms is facilitated by the use of polynucleotides comprising a specific range of fragment sizes. Preparation of such libraries typically involves the fragmentation of large polynucleotides (e.g. cellular genomic DNA) to obtain polynucleotides in the desired size range.

Paired end reads may be used for the sequencing methods and systems disclosed herein. The fragment or insert length is longer than the read length, and sometimes longer than the sum of the lengths of the two reads.

In some aspects, the sample nucleic acid(s) are obtained as genomic DNA, which is subjected to fragmentation into fragments of longer than approximately 50, 100, 200, 300, 400, 500, 600, 700, 800, 900, 1000, 2000, or 5000 base pairs, to which NGS methods can be readily applied. In some aspects, the paired end reads are obtained from inserts of about 100-5000 bp. In some aspects, the inserts are about 100-1000 bp long. These are sometimes implemented as regular short-insert paired end reads. In some aspects, the inserts are about 1000-5000 bp long. These are sometimes implemented as long-insert mate paired reads as described above.

In some aspects, long inserts are designed for evaluating very long sequences. In some aspects, mate pair reads may be applied to obtain reads that are spaced apart by thousands of base pairs. In these aspects, inserts or fragments range from hundreds to thousands of base pairs, with two biotin junction adapters on the two ends of an insert. Then the biotin junction adapters join the two ends of the insert to form a circularized molecule, which is then further fragmented. A sub-fragment including the biotin junction adapters and the two ends of the original insert is selected for sequencing on a platform that is designed to sequence shorter fragments.

Fragmentation can be achieved by any of a number of methods known to those of skill in the art. For example, fragmentation can be achieved by mechanical means including, but not limited to nebulization, sonication and hydroshear. However mechanical fragmentation typically cleaves the DNA backbone at C—O, P—O and C—C bonds resulting in a heterogeneous mix of blunt and 3′-and 5′-overhanging ends with broken C—O, P—O and/C—C bonds (see, e.g., Alnemri and Liwack, J Biol. Chem 265: 17323-17333 [1990]; Richards and Boyer, J Mol Biol 11:327-240 [1965]) which may need to be repaired as they may lack the requisite 5′-phosphate for the subsequent enzymatic reactions, e.g., ligation of sequencing adapters, that are required for preparing DNA for sequencing.

In contrast, cfDNA, typically exists as fragments of less than about 300 base pairs and consequently, fragmentation is not typically necessary for generating a sequencing library using cfDNA samples.

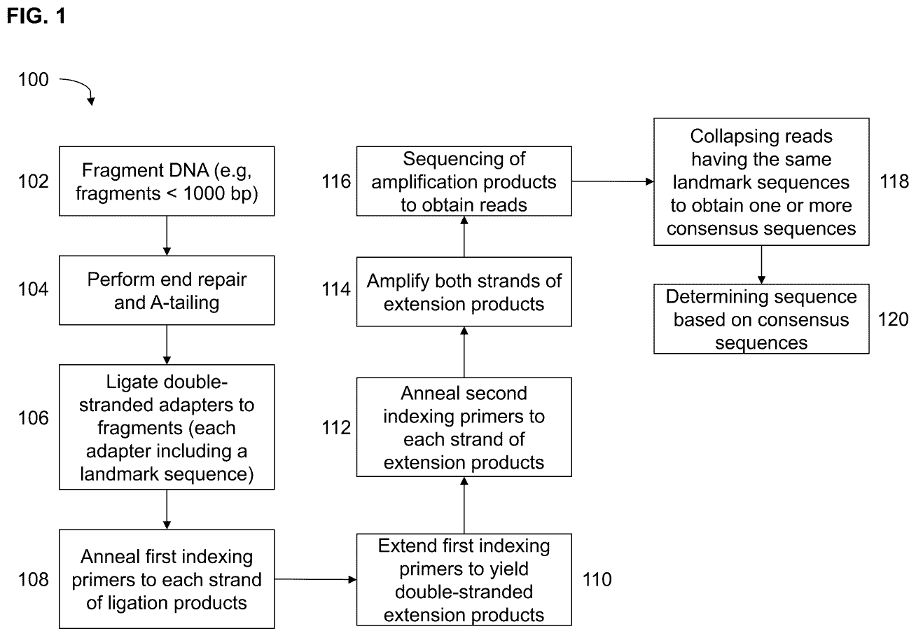

Typically, whether polynucleotides are forcibly fragmented (e.g., fragmented in vitro), or naturally exist as fragments, they are converted to blunt-ended DNA having 5′-phosphates and 3′-hydroxyl. Standard protocols, e.g., protocols for sequencing using, for example, the Illumina platform as described in the example workflow above with reference to FIG. 1, instruct users to end-repair sample DNA, to purify the end-repaired products prior to adenylating or dA-tailing the 3′ ends, and to purify the dA-tailing products prior to the adapter-ligating steps of the library preparation.

Various aspects of methods of sequence library preparation described herein obviate the need to perform one or more of the steps typically mandated by standard protocols to obtain a modified DNA product that can be sequenced by NGS. An abbreviated method (ABB method), a 1-step method, and a 2-step method are examples of methods for preparation of a sequencing library, which can be found in patent application Ser. No. 13/555,037 filed on Jul. 20, 2012, which is incorporated by reference by its entirety.

The methods and apparatus described herein may employ next generation sequencing technology (NGS), which allows massively parallel sequencing. In certain aspects, clonally amplified DNA templates or single DNA molecules are sequenced in a massively parallel fashion within a flow cell (e.g., as described in Volkerding et al. Clin Chem 55:641-658 [2009]; Metzker M Nature Rev 11:31-46 [2010]). The sequencing technologies of NGS include but are not limited to pyrosequencing, sequencing-by-synthesis with reversible dye terminators, sequencing by oligonucleotide probe ligation, and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Examples of sequencing technologies that can be used to obtain the sequence information according to the present method are further described here.

Some sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.), as described below. In addition to the single molecule sequencing performed using sequencing-by-synthesis of Helicos Biosciences, other single molecule sequencing technologies include, but are not limited to, the SMRT™ technology of Pacific Biosciences, the ION TORREN™ technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM). Illustrative sequencing technologies are described in greater detail below.

In some aspects, the disclosed methods involve obtaining sequence information for the nucleic acids in the test sample by massively parallel sequencing of millions of DNA fragments using Illumina's sequencing-by-synthesis and reversible terminator-based sequencing chemistry (e.g. as described in Bentley et al., Nature 6:53-59 [2009]). Template DNA can be genomic DNA, e.g., cellular DNA or cfDNA. In some aspects, genomic DNA from isolated cells is used as the template, and it is fragmented into lengths of several hundred base pairs. In other aspects, cfDNA or circulating tumor DNA (ctDNA) is used as the template, and fragmentation is not required as cfDNA or ctDNA exists as short fragments. For example fetal cfDNA circulates in the bloodstream as fragments approximately 170 base pairs (bp) in length (Fan et al., Clin Chem 56:1279-1286 [2010]), and no fragmentation of the DNA is required prior to sequencing. Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchor oligos. Under limiting-dilution conditions, adapter-modified, single-stranded template DNA is added to the flow cell and immobilized by hybridization to the anchor oligos. Attached DNA fragments are extended and bridge amplified to create an ultra-high density sequencing flow cell with hundreds of millions of clusters, each containing about 1,000 copies of the same template. In one aspect, the randomly fragmented genomic DNA is amplified using PCR before it is subjected to cluster amplification. Alternatively, an amplification-free genomic library preparation is used, and the randomly fragmented genomic DNA is enriched using the cluster amplification alone (Kozarewa et al., Nature Methods 6:291-295 [2009]). In some applications, the templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about tens to a few hundred base pairs are aligned against a reference genome and unique mapping of the short sequence reads to the reference genome are identified using specially developed data analysis pipeline software. After completion of the first read, the templates can be regenerated in situ to enable a second read from the opposite end of the fragments. Thus, either single-end or paired end sequencing of the DNA fragments can be used.

Various aspects of the disclosure may use sequencing by synthesis that allows paired end sequencing. In some aspects, the sequencing by synthesis platform by Illumina involves clustering fragments. Clustering is a process in which each fragment molecule is isothermally amplified. In some aspects, as the example described here, the fragment has two different adapters attached to the two ends of the fragment, the adapters allowing the fragment to hybridize with the two different oligos on the surface of a flow cell lane. The fragment further includes or is connected to two index sequences at two ends of the fragment, which index sequences provide labels to identify different samples in multiplex sequencing. In some sequencing platforms, a fragment to be sequenced from both ends is also referred to as an insert.

In some aspects, a flow cell for clustering in the Illumina platform is a glass slide with lanes. Each lane is a glass channel coated with a lawn of two types of oligos (e.g., P5 and P7′ oligos). Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification and other sequencing methods involving clustering, a strand folds over, and a second adapter region on a second end of the strand hybridizes with the second type of oligos on the flow cell surface. A polymerase generates a complementary strand, forming a double-stranded bridge molecule. This double-stranded molecule is denatured resulting in two single-stranded molecules tethered to the flow cell through two different oligos. The process is then repeated over and over, and occurs simultaneously for millions of clusters resulting in clonal amplification of all the fragments. After bridge amplification, the reverse strands are cleaved and washed off, leaving only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters are sequenced in a massively parallel manner. At the completion of the first read, the read product is washed away.

In the next step of protocols involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flow cell. An index 2 sequence is read in the same manner as index 1. Then an index 2 read product is washed off at the completion of the step.

After reading two indices, read 2 initiates by using polymerases to extend the second flow cell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences. These contiguous sequences are aligned to the reference genome for variant identification.

The sequencing by synthesis example described above involves paired end reads, which is used in many of the aspects of the disclosed methods. Paired end sequencing involves 2 reads from the two ends of a fragment. Paired end reads are used to resolve ambiguous alignments. Paired-end sequencing allows users to choose the length of the insert (or the fragment to be sequenced) and sequence either end of the insert, generating high-quality, alignable sequence data. Because the distance between each paired read is known, alignment algorithms can use this information to map reads over repetitive regions more precisely. This results in better alignment of the reads, especially across difficult-to-sequence, repetitive regions of the genome. Paired-end sequencing can detect rearrangements, including insertions and deletions (indels) and inversions.

Paired end reads may use insert of different length (i.e., different fragment size to be sequenced). As the default meaning in this disclosure, paired end reads are used to refer to reads obtained from various insert lengths. In some instances, to distinguish short-insert paired end reads from long-inserts paired end reads, the latter is specifically referred to as mate pair reads. In some aspects involving mate pair reads, two biotin junction adapters first are attached to two ends of a relatively long insert (e.g., several kb). The biotin junction adapters then link the two ends of the insert to form a circularized molecule. A sub-fragment encompassing the biotin junction adapters can then be obtained by further fragmenting the circularized molecule. The sub-fragment including the two ends of the original fragment in opposite sequence order can then be sequenced by the same procedure as for short-insert paired end sequencing described above. Further details of mate pair sequencing using an Illumina platform is shown in an online publication at the following address, which is incorporated by reference by its entirety: res.illumina.com/documents/products/technotes/technote_nextera_matepair_data_processing.pdf

After sequencing of DNA fragments, sequence reads of predetermined length, e.g., 100 bp, are localized by mapping (alignment) to a known reference genome. The mapped reads and their corresponding locations on the reference sequence are also referred to as tags. In another aspect of the procedure, localization is realized by k-mer sharing and read-read alignment. The analyses of many aspects disclosed herein make use of reads that are either poorly aligned or cannot be aligned, as well as aligned reads (tags). In one aspect, the reference genome sequence is the NCBI36/hg18 sequence, which is available on the World Wide Web at genome.ucsc.edu/cgi-bin/hgGateway?org=Human&db=hg18&hgsid=166260105). Alternatively, the reference genome sequence is the GRCh37/hg19 or GRCh38, which is available on the World Wide Web at genome. ucsc. edu/cgi-bin/hgGateway. Other sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al., Genome Biology 10:R25.1-R25.10 [2009]), or ELAND (Illumina, Inc., San Diego, Calif., USA). In one aspect, one end of the clonally expanded copies of the plasma cfDNA molecules is sequenced and processed by bioinformatics alignment analysis for the Illumina Genome Analyzer, which uses the Efficient Large-Scale Alignment of Nucleotide Databases (ELAND) software.

Other sequencing methods may also be used to obtain sequence reads and alignments thereof. Additional suitable methods are described in U.S. patent application Ser. No. 15/130,668 filed no Apr. 15, 2016, which is incorporated by reference in its entirety.

In some aspects of the methods described herein, the sequence reads are about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. It is expected that technological advances will enable single-end reads of greater than 500 bp enabling for reads of greater than about 1000 bp when paired end reads are generated. In some aspects, paired end reads are used to determine sequences of interest, which comprise sequence reads that are about 20 bp to 1000 bp, about 50 bp to 500 bp, or 80 bp to 150 bp. In various aspects, the paired end reads are used to evaluate a sequence of interest. The sequence of interest is longer than the reads. In some aspects, the sequence of interest is longer than about 100 bp, 500 bp, 1000 bp, or 4000 bp. Mapping of the sequence reads is achieved by comparing the sequence of the reads with the sequence of the reference to determine the chromosomal origin of the sequenced nucleic acid molecule, and specific genetic sequence information is not needed. A small degree of mismatch (0-2 mismatches per read) may be allowed to account for minor polymorphisms that may exist between the reference genome and the genomes in the mixed sample. In some aspects, reads that are aligned to the reference sequence are used as anchor reads, and reads paired to anchor reads but cannot align or poorly align to the reference are used as anchored reads. In some aspects, poorly aligned reads may have a relatively large number of percentage of mismatches per read, e.g., at least about 5%, at least about 10%, at least about 15%, or at least about 20% mismatches per read.

A plurality of sequence tags (i.e., reads aligned to a reference sequence) are typically obtained per sample. In some aspects, at least about 3×10⁶ sequence tags, at least about 5×10⁶ sequence tags, at least about 8×10⁶ sequence tags, at least about 10×10⁶ sequence tags, at least about 15×10⁶ sequence tags, at least about 20×10⁶ sequence tags, at least about 30×10⁶ sequence tags, at least about 40×10⁶ sequence tags, or at least about 50×10⁶ sequence tags of, e.g., 100 bp, are obtained from mapping the reads to the reference genome per sample. In some aspects, all the sequence reads are mapped to all regions of the reference genome, providing genome-wide reads. In other aspects, reads mapped to a sequence of interest.

### III. Compositions and Kits

In an aspect, provided herein are adapters, wherein each adapter comprises a double-stranded region, and wherein each strand of the double-stranded region comprises a landmark sequence comprising a plurality of nucleotide analogs.

The landmark sequence of an adapter can be used, for example, to generate a unique identification sequence through amplification of a landmark sequence-containing nucleic acid. For example, a landmark sequence may be included in an adapter that is ligated onto a template nucleic acid. A polymerase then generates a complement of the adapter-ligated template nucleic acid, generating a complement landmark sequence. The complement landmark sequence does not include the one or more nucleotide analogs of the parental landmark sequence, but instead includes nucleotides complementary to each of the nucleotide analogs. The complement landmark sequence facilitates downstream identification of the original template nucleic acid, and is capable of distinguishing an individual molecule in a large heterogeneous population of molecules.

An adapter including a landmark sequence may be generated using standard oligonucleotide synthesis techniques, for example, by solid-phase synthesis using phosphoramidite building blocks.

In some aspects, adapter molecules can be “Y”-shaped, “U”-shaped, “hairpin” shaped, have a bubble (e.g., a portion of sequence that is non-complimentary), or other features. In other aspects, adapter molecules can comprise a “Y”-shape, a “U”-shaped, a “hairpin” shaped, or a bubble. Certain adapters may comprise modified or non-standard nucleotides, restriction sites, or other features for manipulation of structure or function in vitro. Adapter molecules may ligate to a variety of nucleic acid material having a terminal end. For example, adapter molecules can be suited to ligate to a T-overhang, an A-overhang, a CG-overhang, a multiple nucleotide overhang, a dehydroxylated base, a blunt end of a nucleic acid material and the end of a molecule were the 5′ of the target is dephosphorylated or otherwise blocked from traditional ligation. In other aspects the adapter molecule can contain a dephosphorylated or otherwise ligation-preventing modification on the 5′ strand at the ligation site. In the latter two aspects such strategies may be useful for preventing dimerization of library fragments or adapter molecules.

An adapter sequence can mean a single-strand sequence, a double-strand sequence, a complimentary sequence, a non-complimentary sequence, a partial complimentary sequence, an asymmetric sequence, a primer binding sequence, a flow-cell sequence, a ligation sequence or other sequence provided by an adapter molecule. In particular aspects, an adapter sequence can mean a sequence used for amplification by way of compliment to an oligonucleotide.

In some aspects, the adapters are Y-shaped adapters comprising a single-stranded 5′ arm, a single-stranded 3′ arm, and a double-stranded region comprising the landmark sequence. In some aspects, the single-stranded 3′ arm comprises a capture sequence (see, e.g., FIG. 3A). In some aspects, the 3′ end of the single-stranded region is blocked (e.g., the 3′ end does not comprise a 3′-OH) to prevent extension. In some aspects, the single-stranded 5′ arm comprises a primer binding sequence (e. g, a first primer binding sequence). In some aspects, the double-stranded region of the Y-shaped adapter further comprises an index sequence. In some aspects, the adapters comprise a first plurality of Y-shaped adapters and a second plurality of Y-shaped adapters. In some aspects, the double-stranded region of the first plurality of Y-shaped adapters comprise a first index sequence and the second plurality of Y-shaped adapters comprise a second index sequence. In some aspects, the indeces are used for sample identification (i.e., identification of individual samples from a plurality of samples). In some aspects, the indeces are used for strand identification (i.e., identification and/or tracking of each strand of the double-stranded nucleic acid). In some aspects, the indeces are used for error correction. Exemplary methods for using indeces for error correction are described in, e.g., U.S. Pat. Nos. 10,844,428 and 10,844,429, each of which is incorporated herein by reference.

In some aspects, the double-stranded region of the first plurality of Y-shaped adapters comprise a first landmark sequence and the second plurality of Y-shaped adapters comprise a second landmark sequence.

In some aspects, provided methods and compositions include at least one adapter sequence (e.g., two adapter sequences, one on each of the 5′ and 3′ ends of a nucleic acid material). In some aspects, provided methods and compositions may comprise 2 or more adapter sequences (e.g., 3, 4, 5, 6, 7, 8, 9, 10 or more). In some aspects, at least two of the adapter sequences differ from one another (e.g., by sequence). In some aspects, each adapter sequence differs from each other adapter sequence (e.g., by sequence). In some aspects, at least one adapter sequence is at least partially non-complementary to at least a portion of at least one other adapter sequence (e.g., is non-complementary by at least one nucleotide).

In some aspects, an adapter sequence comprises at least one non-standard nucleotide. In some aspects, a non-standard nucleotide is selected from an abasic site, a uracil, tetrahydrofuran, 8-oxo-7,8-dihydro-2′deoxyadenosine (8-oxo-A), 8-oxo-7,8-dihydro-2′-deoxyguanosine (8-oxo-G), deoxyinosine, 5′nitroindole, 5-Hydroxymethyl-2′-deoxycytidine, iso-cytosine, 5′-methyl-isocytosine, or isoguanosine, a methylated nucleotide, an RNA nucleotide, a ribose nucleotide, an 8-oxo-guanine, a photocleavable linker, a biotinylated nucleotide, a desthiobiotin nucleotide, a thiol modified nucleotide, an acrydite modified nucleotide an iso-dC, an iso dG, a 2′-O-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, an 5′ Hexynyl modified nucleotide, an 5-Octadiynyl dU, photocleavable spacer, a non-photocleavable spacer, a click chemistry compatible modified nucleotide, and any combination thereof.

In some aspects, an adapter sequence comprises a moiety having a magnetic property (i.e., a magnetic moiety). In some aspects this magnetic property is paramagnetic. In some aspects where an adapter sequence comprises a magnetic moiety (e.g., a nucleic acid material ligated to an adapter sequence comprising a magnetic moiety), when a magnetic field is applied, an adapter sequence comprising a magnetic moiety is substantially separated from adapter sequences that do not comprise a magnetic moiety (e.g., a nucleic acid material ligated to an adapter sequence that does not comprise a magnetic moiety).

In some aspects, the adapter is partially double-stranded and is formed by annealing two oligonucleotides corresponding to the two strands. The two strands have a number of complementary base pairs (e.g., 12-17 bp) that allow the two oligonucleotides to anneal at the end to be ligated with a dsDNA fragment. A dsDNA fragment to be ligated on both ends for pair-end reads is also referred to as an insert. Other base pairs are not complementary on the two strands, resulting in a fork-shaped adapter having two floppy overhangs.

In some aspects, the landmark sequence is incorporated into the double-stranded portion of the adapter. Since the two strands of a double-stranded landmark sequence are complementary to each other, the association between the two strands of the double-stranded landmark sequence is inherently reflected by the complementary sequences, and can be established without requiring either a priori or a posteriori information. This information may be used to infer that reads having the two complementary sequences of a double-stranded landmark sequence of an adapter are derived from the same DNA fragment to which the adapter was ligated, but the two complementary sequences of the landmark sequence are ligated to the 3′ end on one strand and the 5′ end on the other strand of the DNA fragment. Therefore, one may collapse not only reads having the same order of two landmark sequences on two ends, but also reads having the reverse order of two complementary sequences on two ends.

In other aspects, the landmark sequence is incorporated into the single-stranded portion of the adapter.

In some aspects, each adapter comprises (i) a first strand comprising, from 5′ to 3′, a first primer binding sequence, a hybridization sequence, and a landmark sequence (i.e., an exogenous landmark sequence); and (ii) a second strand comprising, from 5′ to 3′, a sequence substantially complementary to the landmark sequence, a sequence complementary to the hybridization sequence, and a second primer binding sequence. In some aspects, the landmark sequence and the sequence substantially complementary to the landmark sequence include nucleotide analogs. In some aspects, only one of the landmark sequence and the sequence substantially complementary to the landmark sequence include nucleotide analogs.

In some aspects, each landmark sequence is about 25 base pairs or shorter. In some aspects, each landmark sequence is about 20 base pairs or shorter. In some aspects, each landmark sequence is about 15 base pairs or shorter.

In some aspects, each landmark sequence comprises one or more nucleotide analogs. In some aspects, each landmark sequence comprises 3 or more nucleotide analogs. In some aspects, each landmark sequence comprises 6 or more nucleotide analogs. In some aspects, each landmark sequence comprises 1, 2, 3, 4, 5, 6, or more nucleotide analogs.

In some aspects, the nucleotide analogs (i.e., the nucleotide analogs of the landmark sequence) are not adjacent to each other. In some aspects, each of the nucleotide analogs is separated by at least 2 nucleotides. In some aspects, each of the nucleotide analogs is separated by at least 3 nucleotides. In some aspects, each of the nucleotide analogs is separated by at least 4 nucleotides. In some aspects, each of the nucleotide analogs is separated by at least 5 nucleotides.

In some aspects, the nucleotides analogs comprise one or more degenerate bases. In some aspects, the one or more degenerate bases comprise inosine, the pyrimidine base 6H,8H-3,4-dihydropyrimido[4,5-c][1,2]oxazin-7-one (P), the purine base N⁶-methoxy-2,6-diaminopurine (K), 5-nitroindole, or any combination thereof. In some aspects, the one or more degenerate bases comprise inosine. In some aspects, the one or more degenerate bases comprise the pyrimidine base 6H,8H-3,4-dihydropyrimido[4,5-c][1,2]oxazin-7-one (P). In some aspects, the one or more degenerate bases comprise the purine base N⁶-methoxy-2,6-diaminopurine (K). In some aspects, the one or more degenerate bases comprise 5-nitroindole.

In some aspects, the first strand of the adapter comprises a first landmark sequence and wherein the second strand of the adapter comprises a second landmark sequence. In some aspects, the first landmark sequence and the second landmark sequence are substantially complementary. In some aspects, the first landmark sequence comprises a first exogenous landmark sequence and the second landmark sequence comprises a second exogenous landmark sequence, wherein each of the exogenous landmark sequences comprises one or more nucleotide analogs.

In another aspect, provided herein is a first primer (e.g., a first indexing primer), comprising a sequence complementary to a region 3′ of the landmark sequence in the adapter. In some aspects, the first primer comprises a first index sequence and one or more primer binding sequences.

In some aspects, the first primer comprises an affinity tag at the 5′ end. In some aspects, the affinity tag is a biotin moiety.

In some aspects, the adapters comprise an affinity tag (e.g., a biotin moiety) at a 5′ end (i.e., at a 5′ end of one of the polynucleotide strands of the adapter).

In another aspect, provided herein is a second primer (e.g., a second indexing primer). In some aspects, the second primer is complementary to a portion of the complement of the single-stranded 5′ arm.

In some aspects, each adapter comprises a single-stranded 5′ arm and a single-stranded 3′ arm. In some aspects, the first primer is complementary to a portion of the single-stranded 3′ arm.

In some aspects, the first primer comprises, from 5′ to 3′, a first platform primer binding sequence (e.g., a P7 sequence), a first index sequence (e.g., an i7 sequence), and a sequence complementary to the second primer binding sequence of the adapter. In some aspects, the second primer comprises, from 5′ to 3′, a second platform primer binding sequence (e.g., a P5 sequence), a second index sequence (e.g., an i5 sequence), and a second complementary to the first primer binding sequence of the adapter.

In some aspects, each adapter further comprises a random sequence. In some aspects, the random sequence is between 2 to 8 nucleotides in length. In some aspects, the random sequence is 2, 3, 4, 5, 6, 7, or 8 nucleotides in length.

In some aspects, the adapter attached to the first strand adapter-target nucleic acid sequence and the adapter attached to the second strand adapter-target nucleic acid sequence comprise different landmark sequences.

In some aspects, the adapter is between 25 and 80 nucleotides in length. In some aspects, the adapter is greater than 80 nucleotides in length. In some aspects, the adapter is about 25, about 40, about 50, about 60, about 70, or about 80 nucleotides in length.

In some asepcts, the adapter comprises a 3′ overhang. In some aspects, the adapter is blunt-ended.

In another aspect, the present disclosure provides a kit comprising a plurality of the adapters, a plurality of the first primers, and a plurality of the second primers as described herein. In some aspects, the kit further comprises one or more polymerases. In some aspects, the kit further comprises a solid support comprising an immobilized capture agent. In some aspects, the kit further comprises a solid support comprising an immobilized capture oligonucleotide. In some aspects, the immobilized capture oligonucleotide comprises, from 5′ to 3′, a cleavage site, a primer binding sequence, and a capture sequence complement. In some aspects, the immobilized capture oligonucleotide futher comprises a barcode sequence.

In an aspect is provided a kit. Generally, the kit includes one or more containers providing a composition and one or more additional reagents (e.g., a buffer suitable for polynucleotide extension). The kit may also include a template nucleic acid (DNA and/or RNA), one or more primers, one or more adapters, nucleoside triphosphates (including, e.g., deoxyribonucleotides, ribonucleotides, labeled nucleotides, and/or modified nucleotides), buffers, salts, and/or labels (e.g., fluorophores).

In aspects, the kit includes a sequencing polymerase, and one or more amplification polymerases. In aspects, the sequencing polymerase is capable of incorporating modified nucleotides. In aspects, the polymerase is a DNA polymerase. In aspects, the DNA polymerase is a Pol I DNA polymerase, Pol II DNA polymerase, Pol III DNA polymerase, Pol IV DNA polymerase, Pol V DNA polymerase, Pol (3 DNA polymerase, Pol LI DNA polymerase, Pol X DNA polymerase, Pol o DNA polymerase, Pol a DNA polymerase, Pol 5 DNA polymerase, Pol e DNA polymerase, Pol q DNA polymerase, Pol r DNA polymerase, Pol K DNA polymerase, Pol £ DNA polymerase, Pol y DNA polymerase, Pol 9 DNA polymerase, Pol u DNA polymerase, or a thermophilic nucleic acid polymerase (eg., Therminator y, 9° N polymerase (exo-), Therminator II, Therminator III, or Therminator IX). In aspects, the DNA polymerase is a thermophilic nucleic acid polymerase. In aspects, the DNA polymerase is a modified archaeal DNA polymerase. In aspects, the polymerase is a reverse transcriptase. In aspects, the kit includes a strand-displacing polymerase. In aspects, the kit includes a strand-displacing polymerase, such as a phi29 polymerase, phi29 mutant polymerase or a thermostable phi29 mutant polymerase.

In aspects, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In aspects, the buffered solution can include Tris. With respect to the aspects described herein, the pH of the buffered solution can be modulated to permit any of the described reactions. In some aspects, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other aspects, the buffered solution can have a pH ranging, for example, from about pH 6 to about pH 9, from about pH 8 to about pH 10, or from about pH 7 to about pH 9. In aspects, the buffered solution can include one or more divalent cations. Examples of divalent cations can include, but are not limited to, Mg2+, Mn2+, Zn2+, and Ca2+. In aspects, the buffered solution can contain one or more divalent cations at a concentration sufficient to permit hybridization of a nucleic acid. The kit may also include a flow cell. In aspects, kit includes the solid support and a flow cell carrier (e.g., a flow cell carrier as described in US 2021/0190668, which is incorporated herein by reference for all purposes).

In aspects, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

In another aspect, the present disclosure provides a solid support. In some aspects, the solid support comprises a first immobilized extended capture oligonucleotide and a second immobilized extended capture oligonucleotide. In some aspects, the first immobilized extended capture oligonucleotide comprises a cleavage site, a first primer binding sequence, a capture sequence complement, a first index sequence, a first landmark sequence complement, a second landmark sequence, a second index sequence complement, and a second primer binding sequence complement. In some aspects, the second immobilized extended capture oligonucleotide comprises the cleavage site, the first primer binding sequence, the capture sequence complement, a second index sequence, a second landmark sequence complement, a first landmark sequence, a first index sequence complement, and a second primer binding sequence. In some aspects, the cleavage site is a chemical cleavage site or an enzymatic cleavage site.

### EXAMPLES

### Example 1: Introduction and Applications of Landmark Sequences

Next generation sequencing (NGS) technology has developed rapidly, providing new tools to advance research and science, as well as healthcare and services relying on genetic and related biological information. NGS methods are performed in a massively parallel fashion, affording increasingly high speed for determining biomolecules sequence information. However, many of the NGS methods and associated sample manipulation techniques introduce errors such that the resulting sequences have relatively high error rate, ranging from one error in a few hundred base pairs to one error in a few thousand base pairs. Such error rates are sometimes acceptable for determining inheritable genetic information such as germline mutations because such information is consistent across most somatic cells, which provide many copies of the same genome in a test sample. An error originating from reading one copy of a sequence has a minor or removable impact when many copies of the same sequence are read without error. For instance, if an erroneous read from one copy of a sequence cannot be properly aligned to a reference sequence, it may simply be discarded from analysis. Error-free reads from other copies of the same sequence may still provide sufficient information for valid analyses. Alternatively, instead of discarding the read having a base pair different from other reads from the same sequence, one can disregard the different base pair as resulting from a known or unknown source of error.

However, such error correction approaches do not work well for detecting sequences with low allele frequencies, such as sub-clonal, somatic mutations found in nucleic acids from tumor tissue, circulating tumor DNA, low-concentration fetal cfDNA in maternal plasma, drug-resistant mutations of pathogens, etc. In these examples, one DNA fragment may harbor a somatic mutation of interest at a sequence site, while many other fragments at the same sequence site do not have the mutation of interest. In such a scenario, the sequence reads or base pairs from the mutated DNA fragment might be unused or misinterpreted in conventional sequencing, thereby losing information for detecting the mutation of interest.

Due to these various sources of errors, increasing depth of sequencing alone cannot ensure detection of somatic variations with very low allele frequency (e.g., <1%). Some implementations disclosed herein provide duplex sequencing methods that effectively suppress errors in situations when signals of valid sequences of interest are low, such as samples with low allele frequencies.

Unique molecular indices (UMIs) enable the usage of information from multiple reads to suppress sequencing noise. UMIs, along with contextual information such as alignment positions, allow each read to be traced back to a specific original DNA molecule. Given multiple reads that were produced by the same DNA molecule, computational approaches can be used to separate actual variants (i.e. variants biologically present in the original DNA molecules) from variants artificially introduced via sequencing error. Variants can include, but are not limited to, insertions, deletions, multi-nucleotide variants, single-nucleotide variants, and structural variants. Using this information, the true sequence of the DNA molecules can be inferred. This computational methodology is referred to as read collapsing. This error-reduction technology has several important applications. In the context of cell-free DNA analysis, important variants often occur at extremely low frequencies (i.e. <1%); thus their signal can be drowned out by sequencing errors. UMI-based noise reduction allows for accurate base calling of these low-frequency variants. UMIs and read collapsing can also help identify PCR duplicates in high-coverage data, enabling more accurate variant frequency measurements.

Adapters can include exogneous landmark sequences that allow one to determine which strand of the DNA fragment the reads are derived from. Exogenous landmarks sequences include a plurality of nucleotide analogs. Upon amplification by a polymerase, the complement of the exogenous landmark sequence includes a nucleotide composition that does not include nucleotide analogs (i.e., the amplification reaction was performed in the presence of only native nucleotides). Some aspects take advantage of this to determine a first consensus sequence for reads derived from one strand of the DNA fragment, and a second consensus sequence for the complementary strand. In many aspects, a consensus sequence includes the nucleotides detected in all or a majority of reads while excluding nucleotides appearing in few of the reads. Different criteria of consensus may be implemented. The process of combining reads based on landmark sequences or alignment locations to obtain a consensus sequence is also referred to as “collapsing” the reads. Using exogenous landmark sequences, endogenous landmark sequences, and/or alignment locations, one can determine that reads for the first and second consensus sequences are derived from the same double stranded fragment. Therefore, in some aspects, a third consensus sequence is determined using the first and second consensus sequences obtained for the same DNA molecule/fragment, with the third consensus sequence including nucleotides common for the first and second consensus sequences while excluding those inconsistent between the two. In other aspects, only one consensus sequence is directly obtained by collapsing all reads derived from both strands of the same fragment, instead of by comparing the two consensus sequences obtained from the two strands. Finally, the sequence of the fragment may be determined from the third or the only one consensus sequence, which includes base pairs that are consistent across reads derived from both strands of the fragment.

In some aspects, the methods described herein combines different types of indices to determine the source polynucleotide on which reads are derived. For example, the method may use both exogenous and endogenous landmark sequences to identify reads deriving from a single DNA molecule. By using a second form of UMI, in addition to the physical UMI, the physical UMIs may be shorter than when only physical UMIs are used to determine the source polynucleotide. This approach has minimal impact on library prep performance, and does not require extra sequencing read length.

Non-limiting applications of the disclosed methods include, for example: error suppression for somatic mutation detection, such as detection of mutation with less than 0.1% allele frequency is highly critical in liquid biopsy of circulating tumor DNA; correcting prephasing, phasing and other sequencing errors to achieve high quality long reads (e.g., 1×1000 bp); decreasing cycle time for fixed read length, and correcting increased phasing and prephasing by this method; and quantifying or counting nucleic acid fragments relating to a sequence of interest.

### Example 2: Generating Uniquely-Labeled Nucleic Acid Strands with Landmark Sequences

FIG. 1 illustrates a flow chart of an exemplary workflow 100 using landmark sequences to sequence nucleic acid fragments. Workflow 100 is illustrative of only some implementations. It is understood that some implementations employ workflows with additional operations not illustrated here, while other implementations may skip some of the operations illustrated here. For instance, some implementations do not require operation 102 and/or operation 104.

Operation 102 provides fragments of double-stranded DNA. The DNA fragments may be obtained by fragmenting genomic DNA, collecting naturally fragmented DNA (e.g., cfDNA or ctDNA), or synthesizing DNA fragments from RNA, for example. In some implementations, to synthesize DNA fragments from RNA, messenger RNA or noncoding RNA is first purified using polyA selection or depletion of ribosomal RNA, then the selected mRNA is chemically fragmented and converted into single-stranded cDNA using random hexamer priming. A complementary strand of the cDNA is generated to create a double-stranded cDNA that is ready for library construction. To obtain double stranded DNA fragments from genomic DNA (gDNA), input gDNA is fragmented, e.g., by hydrodynamic shearing, nebulization, enzymatic fragmentation, etc., to generate fragments of appropriate lengths, e.g., about 1000 bp, 800 bp, 500, or 200 bp. For instance, nebulization can break up DNA into pieces less than 800 bp in short periods of time. This process generates double-stranded DNA fragments.

In some implementations, fragmented or damaged DNA may be processed without requiring additional fragmentation. For instance, formalin-fixed, paraffin embedded (FFPE) DNA or certain cfDNA are sometimes fragmented enough that no additional fragmentation step is required.

Thousands to millions of double-stranded fragments of a sample can be prepared simultaneously in the workflow. DNA fragmentation by physical methods produces heterogeneous ends, comprising a mixture of 3′ overhangs, 5′ overhangs, and blunt ends. The overhangs will be of varying lengths and ends may or may not be phosphorylated.

If DNA fragments are produced by physical methods, workflow 100 proceeds to perform end repair operation 104, which produces blunt-end fragments having 5′-phosphorylated ends. In some implementations, this step converts the overhangs resulting from fragmentation into blunt ends using T4 DNA polymerase and Klenow enzyme. The 3′ to 5′ exonuclease activity of these enzymes removes 3′ overhangs and the 5′ to 3′ polymerase activity fills in the 5′ overhangs. In addition, T4 polynucleotide kinase in this reaction phosphorylates the 5′ ends of the DNA fragments.

After end repairing, operation 104 includes a step of A-tailing to adenylate 3′ ends of the fragments, which is also referred to as dA-tailing, because a single dATP is added to the 3′ ends of the blunt fragments to prevent them from ligating to one another during the adapter ligation reaction.

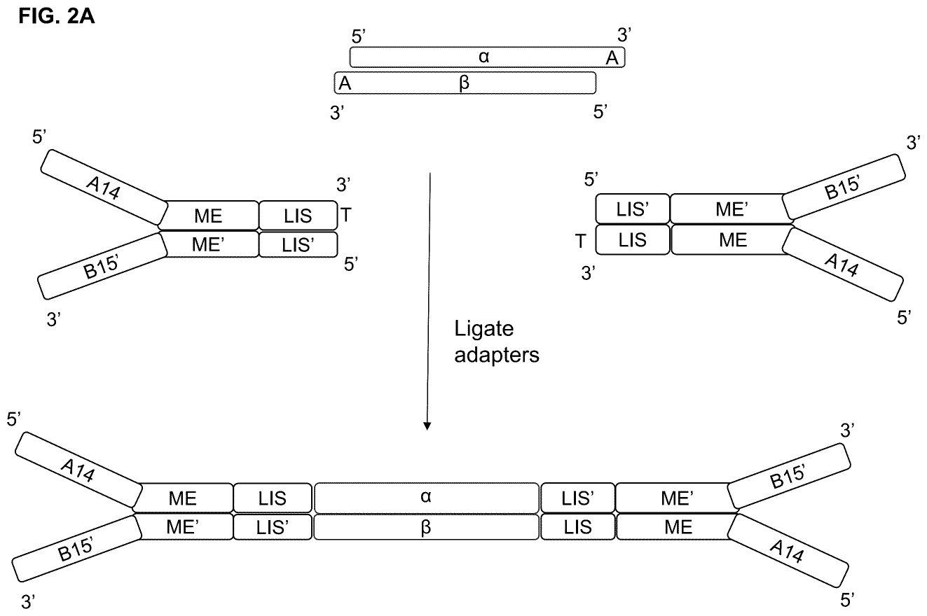

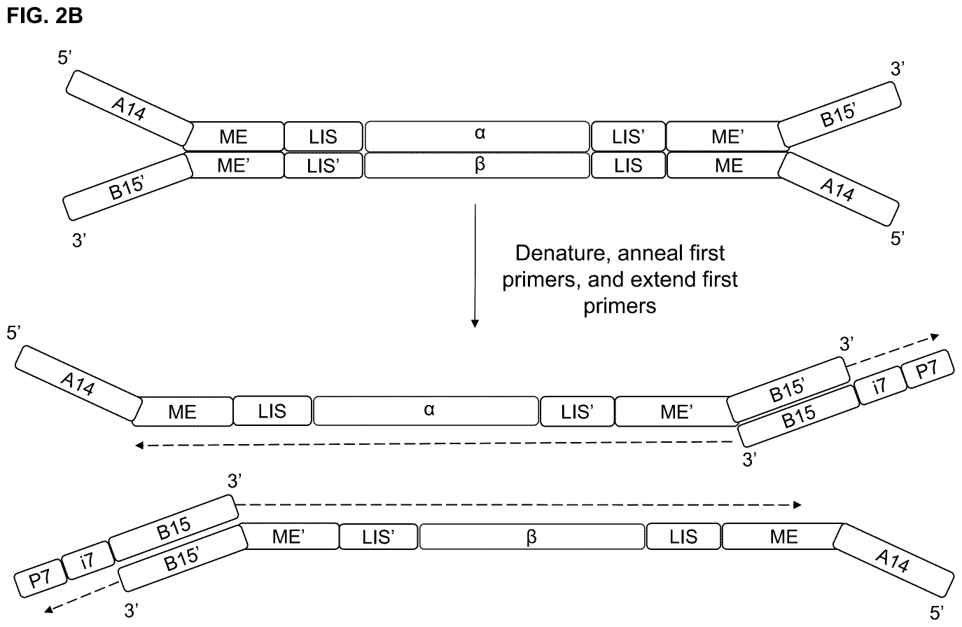

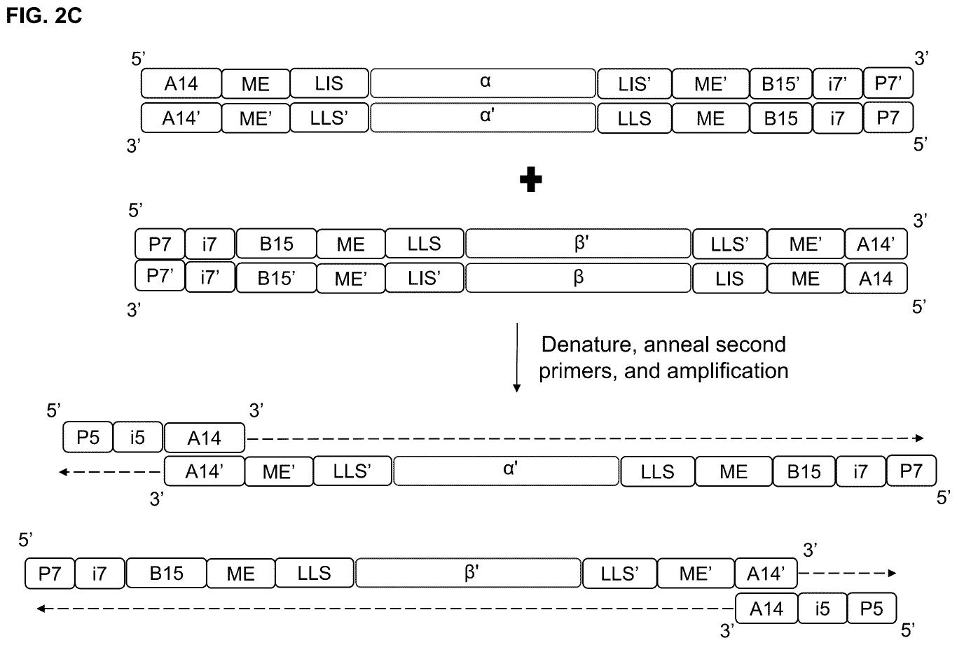

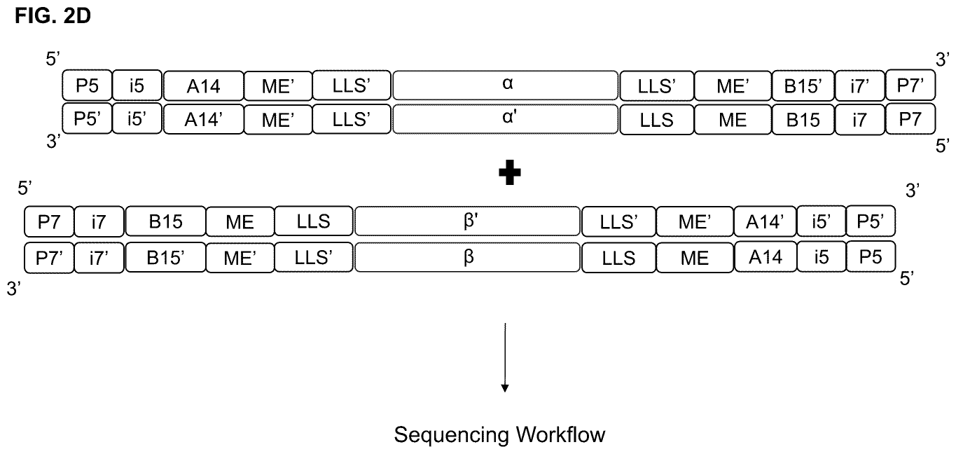

After adenylating 3′ ends, workflow 100 proceeds to operation 106 to ligate partially double stranded adapters to both ends of the fragments. In some aspects, the adapters used in a reaction include different landmark sequence to associate sequence reads to a single source polynucleotide, which may be a single-or double-stranded DNA fragment. This is further illustrated in FIG. 2A. FIG. 2A shows the step of ligating landmark sequence (LIS)-containing adapters to an end-repaired and A-tailed double-stranded nucleic acid fragment. The top strand of the double-stranded nucleic acid fragment is labeled with as ‘α’and the bottom strand as ‘β’.

After adapter ligation, first indexing primers are annealed to each strand of the ligation products in operation 108, followed by linear amplification of both strand of the ligation products in operation 110. Prior to annealing the first indexing primers, the double-stranded ligation products may be denatured, for example, by chemical or thermal denaturation. After the first indexing primers have been annealed, a thermostable polymerase may be used to generate a complement of each template strand that the indexing primer is hybridized to. By linear amplification it is meant that no additional denaturation and rehybridization of primers is performed during operation 110. FIG. 2B shows the steps of denaturing the adapter-ligated double-stranded nucleic acid, annealing first indexing primers, and extending the first indexing primers. FIG. 2C shows the product of the first indexing primer extension, wherein the complement of the landmark sequence (LIS) is shown as LLS′, and the complement of LIS′ is shown as LLS.

After operation 110, unincorporated (i.e., unextended) first primers may be removed by single-stranded nuclease treatment, chromatography, and/or ultrafiltration. In some cases, the first indexing primers comprise an affinity tag (e.g, a biotin tag) to allow for subsequent immobilization and purification of the extension products. In some cases, the adapters ligated in operation 106 comprise an affinity tag to allow for subsequent immobilization and purification of the extension products. In some cases, the adapters ligated in operation 106 comprise a capture sequence that is complementary to an immobilized capture oligonucleotide, and which may be used for subsequent immobilization and purification of the double-stranded nucleic acids.

After first indexing primer extension, second indexing primers are annealed to each strand of the first indexing primer extension products in operation 112, followed by amplification (e.g., linear or exponential amplification), in operation 114. FIG. 2D shows the product of the amplification reaction, which may subsequently be used in a sequencing workflow to obtain read as in operation 116. For example, the amplification products may be subjected to solid phase amplification and immobilization on an Illumina flow cell and subsequent sequencing-by-synthesis. Other sequencing workflows are known in the art.

Both the first and second indexing primers include i5 and i7 index sequences. The index sequences serving as a unique identifier for each specific sample. Typically, dual indexes are used during library preparation, with one index on each side of the sequence. Additionally, both the first and second indexing primers include P5 and P7 sequences, which allow the library amplification products to bind and generate amplification clusters on a flow cell surface prior to a sequencing process, for example.

Once sequence reads are obtained, reads having the same landmark sequences (e.g., exogenous and/or endogenous landmark sequence) are collapsed to obtain one or more consensus sequences as in operation 118. A consensus sequence includes nucleotide bases that are consistent or meet a consensus criterion across reads in a collapsed group. Exogenous landmark sequences, endogenous landmark sequences, and position information may be combined in various ways to collapse reads to obtain consensus sequences for determining the sequence of a fragment or at least a portion thereof. In some aspects, exogenous landmark sequences are combined with endogenous landmark sequences to collapse reads. In other aspects, exogenous landmark sequences and read positions are combined to collapse reads. Read position information may be obtained by various techniques using different position measurements, e.g., genomic coordinates of the reads, positions on a reference sequence, or chromosomal positions. In further implementations, exogenous landmark sequences, endogenous landmarks sequences, and read positions are combined to collapse reads.

Finally, workflow 100 uses the one or more consensus sequences to determine the sequence of the nucleic acid fragment from the sample. See operation 120. This may involve determining the nucleic acid fragment's sequence as the third consensus sequence or the single consensus sequence described above.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

1. A method for amplifying a double-stranded nucleic acid molecule, the method comprising:
a. attaching adapters to both ends of a target double-stranded nucleic acid molecule, thereby generating a double-stranded nucleic acid molecule comprising a first strand adapter-target nucleic acid sequence and a second strand adapter-target nucleic acid sequence, wherein each adapter comprises a double-stranded region, and wherein each strand of the double-stranded region comprises a landmark sequence comprising a plurality of nucleotide analogs;
b. annealing a first primer to each of the first strand adapter-target nucleic acid sequence and the second strand adapter-target nucleic acid sequence;
c. extending the annealed first primers with a polymerase, thereby generating a first double-stranded extension product comprising a complement of the first strand adapter-target nucleic acid, and a second double-stranded extension product comprising a complement of the second strand adapter-target nucleic acid sequence, wherein each of the complement of the first strand adapter-target nucleic acid sequence and complement of the second strand adapter-target nucleic acid sequence comprises a landmark sequence complement;
d. annealing a second primer to each of the complement of the first strand adapter-target nucleic acid sequence and the complement of the second strand adapter-target nucleic acid sequence; and
e. amplifying the complement of the first strand adapter-target nucleic acid sequence and the complement of the second strand adapter-target nucleic acid sequence with a polymerase, thereby generating a plurality of first amplification products and a plurality of second amplification products.

2-13. (canceled)

14. The method of claim 1, wherein the adapters comprise an affinity tag at a 5′ end.

15. (canceled)

16. The method of claim 14, wherein the method further comprises, after a, contacting the double-stranded nucleic acid molecule of step a to a solid support comprising an immobilized capture agent, wherein the immobilized capture agent binds to the affinity tag of the adapters.

17-19. (canceled)

20. The method of claim 1, wherein the plurality of first amplification products comprises a sequence of the first strand of the target double-stranded nucleic acid molecule, and complement thereof, and wherein the plurality of second amplification products comprises the second strand of the target double-stranded nucleic acid molecule, and complement thereof.

21. The method of claim 1, wherein the landmark sequence complement does not comprise nucleotide analogs.

22. The method of claim 1, wherein each landmark sequence comprises 3 or more or 6 or more nucleotide analogs.

23-26. (canceled)

27. The method of claim 22, wherein the nucleotide analogs comprise one or more degenerate bases.

28. The method of claim 27, wherein the one or more degenerate bases comprise inosine, the pyrimidine base 6H,8H-3,4-dihydropyrimido[4,5-c] [1,2]oxazin-7-one (P), the purine base N⁶-methoxy-2,6-diaminopurine (K), 5-nitroindole, or any combination thereof.

29-31. (canceled)

32. The method of claim 1, wherein the first strand of the adapter comprises a first landmark sequence and wherein the second strand of the adapter comprises a second landmark sequence.

33. (canceled)

34. The method of claim 32, wherein the first landmark sequence consists of a first exogenous landmark sequence and the second landmark sequence consists of a second exogenous landmark sequence, wherein each of the exogenous landmark sequences comprises one or more nucleotide analogs.

35. (canceled)

36. The method of claim 34, wherein the plurality of first amplification products is related to the plurality of second amplification products by the first exogenous landmark sequence, the first endogenous landmark sequence, or a combination thereof.

37-39. (canceled)

40. The method of claim 1, wherein the adapters are Y-shaped adapters comprising a single-stranded 5′ arm, a single-stranded 3′ arm, and a double-stranded region comprising the landmark sequence.

41-43. (canceled)

44. The method of claim 40, wherein the first strand adapter-target nucleic acid sequence comprises, from 5′ to 3′, a first primer binding sequence, a first index sequence, a first landmark sequence complement, a target nucleic acid sequence, a second landmark sequence, a second index sequence complement, and a capture sequence, and wherein the second strand adapter-target nucleic acid sequence comprises, from 5′ to 3′, a first primer binding sequence, a second index sequence, a second landmark sequence complement, a target nucleic acid sequence complement, a first landmark sequence, a first index sequence complement, and the capture sequence.

45. The method of claim 44, wherein the method further comprises hybridizing the double-stranded nucleic acid molecule to a solid support comprising immobilized capture oligonucleotides, wherein the immobilized capture oligonucleotides are complementary to the capture sequence of the Y-shaped adapter, thereby forming double-stranded nucleic acid molecules complexes.

46-65. (canceled)

66. The method of claim 1, further comprising (f) sequencing the plurality of first amplification products, thereby generating a plurality of first sequence reads, and sequencing the plurality of second amplification products, thereby generating a plurality of second sequence reads.

67. The method of claim 66, further comprising (g) comparing at least one sequence of the plurality of first sequence reads with at least one sequence obtained from the plurality of second sequence reads, thereby generating a consensus sequence of the double-stranded target nucleic acid molecule.

68. The method of claim 67, wherein generating the consensus sequence comprises grouping the at least one sequence of the plurality of first sequence reads with at least one sequence obtained from the plurality of second sequence reads is based at least on the landmark sequence, or complement thereof.

69-75. (canceled)

76. A kit comprising a plurality of the adapters, a plurality of the first primers, and a plurality of the second primers of claim 1.

77-81. (canceled)

82. A solid support comprising a first immobilized extended capture oligonucleotide and a second immobilized extended capture oligonucleotide, wherein the first immobilized extended capture oligonucleotide comprises a cleavage site, a first primer binding sequence, a capture sequence complement, a first index sequence, a first landmark sequence complement, a second landmark sequence, a second index sequence complement, and a second primer binding sequence complement.

83. The solid support of claim 82, wherein the second immobilized extended capture oligonucleotide comprises the cleavage site, the first primer binding sequence, the capture sequence complement, a second index sequence, a second landmark sequence complement, a first landmark sequence, a first index sequence complement, and a second primer binding sequence.

84. (canceled)
